(19) United States
(12) Patent Application Publication
(10) Pub. No.: US 2026/0227404 A1
(43) Pub. Date: Aug. 6, 2026

(54) METHOD OF DETECTING DISEASE-ASSOCIATED MARKERS IN A LIQUID BIOPSY SAMPLE USING EXTRACELLULAR VESICLES

(71) Applicant: The General Hospital Corporation, Boston, MA (US)

(72) Inventor: Hyungsoon Im, Boston, MA (US)

(21) Appl. No.: 19/149,398

(22) PCT Filed: Jan. 18, 2024
(86) PCT No.: PCT/US2024/012015

§ 371 Date: Jul. 18, 2025

Related U.S. Application Data
(60) Provisional application No. 63/480,554, filed on Jan. 19, 2023.

(51) Int. Cl. G01N 33/57525; G01N 33/543; G01N 33/575

(52) U.S. Cl. CPC G01N 33/57525; G01N 33/54373; G01N 33/57575; G01N 33/5759; G01N 2333/4725; G01N 2333/71

(57) ABSTRACT

The present disclosure relates to devices (e.g., nano-plasmonic chips carrying arrays of nanowells) and methods for the detection of cancer (e.g., CCA). Specifically, extracellular vesicles (EVs) can be captured in the nanowells of the nano-plasmonic chips and are then subject to multi-channel imaging that detects the presence and/or level of biomarkers associated with cancers such as CCA. Accordingly, in one aspect, provided herein are methods of detecting extracellular vesicles (EVs) from cholangiocarcinoma (CCA) cells in a sample, the method comprising: obtaining a sample, e.g., from a subject; isolating EVs from the sample; capturing EVs isolated from the sample on a nano-plasmonic chip bearing an array of nanostructures coated with a layer of gold; and detecting the presence and/or levels of one or more of MUC 1, EGER, and EpCAM, wherein a higher expression level of one or more of MUC1, EGFR, and EpCAM compared to a corresponding reference level indicates that the sample includes EVs from CCA cells.

### CLAIM OF PRIORITY

This application claims the benefit of U.S. Provisional Application Ser. No. 63/480,554, filed on Jan. 19, 2023. The entire content of the foregoing is incorporated herein by reference.

This invention was made with government support under Grant Nos. R01GM138778 and R21CA217662 awarded by The National Institutes of Health. The Government has certain rights in the invention.

### TECHNICAL FIELD

Provided herein are methods, devices, and systems for detecting disease-associated markers in a biological sample (e.g., liquid biopsy sample). Specifically, the disclosure relates to methods of enhancing electromagnetic radiation, e.g., optical, signals of extracellular vesicles, and more particularly, to small numbers of extracellular vesicles (EVs).

### BACKGROUND

Cholangiocarcinoma (CCA) is a fatal disease often detected late in unresectable stages. Currently, there are no effective diagnostic methods or biomarkers to detect CCA early with high confidence.

Extracellular vesicles (EVs) present new opportunities as circulating biomarkers for cancers, cardiovascular, neurodegenerative, and infectious diseases, among others. These cell-derived phospholipid vesicles are abundantly present in various bodily fluids (e.g., blood, cerebrospinal fluid, urine, and saliva). More importantly, they carry a variety of biomolecules (lipids, proteins, and genetic materials) originating from their parental cells, which can be harnessed as a minimally invasive means to probe the molecular status of their cellular origins.

In further exploiting EVs' potential and accelerating their clinical adaptation, a critical unmet need is to develop sensitive, robust, and standardized assays that can determine the composition and molecular profiles of EVs in clinical samples. However, their unique sizes (50-1000 nm) impose technical challenges in conventional analytical methods, which often lead to variable findings. Flow cytometry, for example, often underestimates EV counts; small EVs (i.e., exosomes, typically smaller than 200 nm) can be missed due to their weak light scattering, or a group of vesicles can be counted as a single event. Conventional methods, particularly for protein analyses (e.g., Western blotting, enzyme-linked immunosorbent assay/ELISA), consume large amounts of samples and involve time-consuming and extensive processing steps, making them impractical for clinical scenarios.

Therefore, there is a need in the art for methods of analyzing tumor-derived extracellular vesicles (tEVs) harvested from liquid biopsies (e.g., from CCA patients) and methods of detecting CCA.

### SUMMARY

The present disclosure relates to devices (e.g., nano-plasmonic chips carrying arrays of nanowells) and methods for the detection of cancer (e.g., CCA). Specifically, extracellular vesicles (EVs) can be captured in the nanowells of the nano-plasmonic chips and are then subject to multi-channel imaging that detects the presence and/or level of biomarkers associated with cancers such as CCA.

Accordingly, in one aspect, provided herein are methods of detecting extracellular vesicles (EVs) from cholangiocarcinoma (CCA) cells in a sample, the method comprising: obtaining a sample, e.g., from a subject; isolating EVs from the sample: capturing EVs isolated from the sample on a nano-plasmonic chip bearing an array of nanostructures coated with a layer of gold; and detecting the presence and/or levels of one or more of MUC1, EGFR, and EpCAM, wherein a higher expression level of one or more of MUC1, EGFR, and EpCAM compared to a corresponding reference level indicates that the sample includes EVs from CCA cells.

In some embodiments, the method further comprises immunolabeling the captured EVs using a binding ligand.

In some embodiments, the binding ligand is an antibody or a nucleic acid probe.

In some embodiments, the antibodies are fluorophore-conjugated fluorescence primary antibodies or unconjugated primary antibodies with fluorophore-conjugated secondary antibodies labeling the primary antibody.

In some embodiments, EV membrane permeabilization is carried out for antibodies targeting proteins inside the EVs.

In some embodiments, the fluorophore is selected from Cy3, Cy5, AF367, AF405, AF488, AF555, AF647, DL755, and Cy7.

In some embodiments, the nucleic acid probe is an aptamer, a molecular beacon, or a DNA strand.

In some embodiments, the presence and/or levels of one or more of MUC1. EGFR and EpCAM is determined by the fluorescence signal of the fluorophore-attached ligands.

In some embodiments, the method further comprises imaging the plasmonic chip by plasmon-enhanced multi-channel imaging.

In some embodiments, the plasmon-enhanced multi-channel imaging comprises amplifying fluorescent signals from the plasmonic gold nanowell arrays at multiple wavelengths to improve single EV detection sensitivity.

In some embodiments, the plasmon-enhanced multi-channel imaging comprises co-localization analysis from multiple imaging channels and co-existing marker analysis on a single EV level.

In some embodiments, the corresponding reference level is a level of expression of MUC1, EGFR, and EpCAM on EVs from a healthy subject.

In some embodiments, the nanostructures comprise nanoholes, nanorods, nanodisks, nanowells, nanosquares, nanopillars, or nanogrooves.

In some embodiments, the sample comprises bile or a liquid containing cells obtained from the bile duct.

In some embodiments, the nanostructures are nanowells.

In some embodiments, the method comprises detecting the presence and/or levels of EpCAM.

In some embodiments, the method comprises detecting the presence and/or levels of two or more of MUC1, EGFR, and EpCAM.

In some embodiments, the method comprises detecting the presence and/or levels of both EGFR and EpCAM.

In some embodiments, the method comprises detecting the presence and/or levels of all three of MUC1, EGFR, and EpCAM.

In one aspect, provided herein are nanowell nano-plasmonic chips for detecting target extracellular vesicles (EVs) in a sample, comprising a substrate; a plurality of nanowells arranged to form a periodic array of nanowells on the substrate, wherein the periodic array of nanowells is arranged and dimensioned to amplify one or more optical signals of electromagnetic radiation emitted, scattered, or reflected by EVs bound to the nanowells and/or EVs bound to the substrate near the nanowells, or to amplify one or more optical signals of electromagnetic radiation emitted, scattered, or reflected by reporter groups attached to the EVs; and one or more affinity ligands fixed on or adjacent to the nanowells, wherein the affinity ligands selectively bind to target EVs to bind the target EVs to the nanowells or to the substrate adjacent to the nanowells.

In some embodiments, the one or more affinity ligands comprise polyethylene glycol (PEG).

In some embodiments, the optical signal comprises a fluorescent signal, a Raman signal, or dark-field scattering.

In some embodiments, the nanowells are coated with a layer of gold (Au).

In one aspect, provided herein are methods of producing a nanowell nano-plasmonic chip, the method comprising obtaining a semiconductor wafer with a silicon nitride (SiNx) surface layer; applying a photoresist coating on top of the SiNx layer; patterning a periodic nanowell array using lithography; and depositing a layer of metal on the top of the patterned arrays, thereby producing a nanowell nano-plasmonic chip.

In some embodiments, the metal layer is a layer comprising one or more of gold, silver, copper, aluminum, or platinum, and their alloys, wherein the layer is between 50 nm and 500 nm thick.

In some embodiments, the metal layer is a layer comprising one or more of gold and silver, wherein the layer is between 50 nm and 500 nm thick.

In some embodiments, the semiconductor wafer comprises a silicon wafer.

In some embodiments, the method further comprises applying an anti-reflection coating (ARC) on top of the photoresist coating.

In some embodiments, the method further comprises applying an anti-reflection coating (ARC) on top of the SiNx layer and below the photoresist coating.

In one aspect, provided herein are nano-plasmonic chips for use in detecting extracellular vesicles (EVs) from cholangiocarcinoma (CCA) cells in a sample, wherein the detection comprises: obtaining a sample, e.g., from the subject; isolating EVs from the sample; capturing EVs isolated from the sample on a nano-plasmonic chip bearing an array plurality of nanostructures coated with a layer of gold; and detecting the presence and/or levels of one or more of MUC1, EGFR and EpCAM, wherein a higher expression level of one or more of MUC1, EGFR and EpCAM compared to a corresponding reference level indicates that the subject has CCA.

In some embodiments, the detection further comprises immunolabeling the captured EVs using a binding ligand.

In some embodiments, the binding ligand is an antibody or a nucleic acid probe.

In some embodiments, the antibodies are fluorophore-conjugated fluorescence primary antibodies or unconjugated primary antibodies with fluorophore-conjugated secondary antibodies labeling the primary antibody.

In some embodiments, EV membrane permeabilization is carried out for antibodies targeting proteins inside the EVs.

In some embodiments, the fluorophore is selected from Cy3, Cy5, AF367, AF405, AF488, AF555, AF647, DL755, and Cy7.

In some embodiments, the nucleic acid probe is an aptamer, a molecular beacon, or a DNA strand.

In some embodiments, the presence and/or levels of one or more of MUC1. EGFR and EpCAM are determined by the fluorescence signal of the fluorophore-attached ligands.

In some embodiments, the detection further comprises imaging the plasmonic chip by plasmon-enhanced multi-channel imaging.

In some embodiments, the plasmon-enhanced multi-channel imaging comprises amplifying fluorescent signals from the plasmonic gold nanowell arrays at multiple wavelengths to improve single EV detection sensitivity.

In some embodiments, the plasmon-enhanced multi-channel imaging comprises co-localization analysis from multiple imaging channels and co-existing marker analysis on a single EV level.

In some embodiments, the corresponding reference level is a level of expression of MUC1, EGFR, and EpCAM on EVs from a healthy subject.

In some embodiments, the nanostructures comprise nanoholes, nanorods, nanodisks, nanowells, nanosquares, nanopillars, or nanogrooves.

In some embodiments, the sample comprises bile or a liquid containing cells obtained from the bile duct.

Certain terms employed within this document are collected here. Unless stated otherwise, or implicit from context, the following terms and phrases include the meanings provided below to aid in describing particular embodiments.

As used herein the term “comprising” or “comprises” is used in reference to compositions, methods, and respective component(s) thereof, that are useful to an embodiment, yet open to the inclusion of unspecified elements, whether useful or not.

The singular terms “a,” “an,” and “the” comprise plural referents unless context clearly indicates otherwise. Similarly, the word “or” is intended to include “and” unless the context clearly indicates otherwise. The abbreviation, “e.g.” is used herein to indicate a non-limiting example.

The term “periodicity,” as used herein, refers to a recurrence or repetition of a nanostructure (e.g., nanowell) at regular intervals by their positioning on a substrate and/or in an array. The term “periodic” as used herein therefore refers to the regular predefined pattern of nanostructures with respect to each other, such as a lattice or other repeating unit configuration.

“Surface plasmon resonance (SPR),” as used herein, refers to the physical phenomenon in which incident light stimulates collective resonant electron oscillations at planar metal surfaces, and in particular, at interfaces between negative and positive permittivity materials stimulated by incident light.

The term “localized surface plasmon resonance (LSPR)” refers to surface plasmon resonance of nanometer-sized structures, such as a metallic nanoparticle. The oscillating electrons produce strong electromagnetic fields in the (non-conducting) ambient medium near the surface of the metal.

As used herein, the terms “surface plasmons,” “surface plasmon polaritons,” and “plasmons” refer to the collective oscillations of free electrons at plasmonic surfaces, such as metals. These oscillations result in self-sustaining, surface electromagnetic waves that propagate in a direction parallel to the metal/dielectric (or metal/vacuum) interface. Because the wave is on the boundary of a metal and the external medium (air or water, for example), these oscillations are very sensitive to any refractive index change of this boundary, such as, for example, the adsorption of a molecular target, such as an EV, to the metal surface. Additionally, the electromagnetic field strength decays exponentially from the metal surface to the surrounding environment (e.g., vacuum or dielectric). A maximum value of the electromagnetic field strength can be found at the metal/dielectric or metal/vacuum interface.

As used herein, the term “sample” means any biological or other fluids that may contain one or more extracellular vesicles (e.g., exosomes). Such biological fluids include, without limitation, fluids derived from or containing cells, organisms (bacteria, viruses), lysed cells or organisms, cellular extracts, nuclear extracts, components of cells or organisms, extracellular fluid, media in which cells or organisms are cultured in vitro, blood, plasma, serum, gastrointestinal secretions, ascites, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, pleural fluid, nipple aspirates, breast milk, external sections of the skin, respiratory, intestinal, and genitourinary tracts, and prostatic fluid. A sample can be a viral or bacterial sample, a sample obtained from an environmental source, such as a body of polluted water, an air sample, or a soil sample, as well as a food industry sample.

A “biological sample” is derived or obtained from a living organism. The organism can be a whole organism or can be cells or organs grown in culture. In one embodiment, a “biological sample” also refers to a cell or population of cells or a quantity of tissue or fluid from a subject. Most often, a sample has been removed from a subject. Often, a “biological sample” will contain cells from a subject, but the term can also refer to non-cellular biological material, such as non-cellular fractions of blood, saliva, or urine. In one embodiment, a biological sample is from a resection, bronchoscopic biopsy, or core needle biopsy of a primary, secondary, or metastatic tumor, e.g., breast, ovarian, pancreatic, biliary tract, colorectal, glioblastoma, lung tumors or a cell block from pleural fluid. In addition, fine needle aspirate biological samples are also useful.

Biological samples also include explants and primary and/or transformed cell cultures derived from patient tissues. A biological sample can be provided by removing a sample of cells from a subject, but can also be accomplished by using previously isolated cells or cellular extracts (e.g., isolated by another person, at another time, and/or for another purpose). Archival tissues, such as those having treatment or outcome history may also be used. Biological samples include, but are not limited to, tissue biopsies, scrapes (e.g., buccal scrapes), whole blood or other bodily fluids, such as bile, plasma, serum, urine, saliva, cell culture, urine, ascites, pleural fluid, or cerebrospinal fluid.

The samples analyzed by the systems and methods described herein may have been processed, e.g., by purification or enrichment of EVs contained therein, prior to analysis.

As used herein, an “extracellular vesicle” (“EV”) refers to a naturally occurring or synthetic vesicle that includes a cavity inside. The EVs comprise a lipid bilayer membrane enclosing contents of the internal cavity. An EV can include, but is not limited to, an ectosome, a microvesicle, a microparticle, an exosome, an oncosome, an apoptotic body, a liposome, a vacuole, a lysosome, a transport vesicle, a secretory vesicle, a gas vesicle, a matrix vesicle, or a multivesicular body. An EV has a dimension of up to about 10 microns, but are typically about 1000 nm or less.

Exosomes and microvesicles are types of EVs, and can be shed by eukaryotic cells, or budded off of the plasma membrane, to the exterior of the cell. These membrane-bound vesicles are heterogeneous in size with diameters ranging from about 10 nm to about 5000 nm. The methods and compositions described herein are equally applicable for microvesicles of all sizes.

In some embodiments, the term “exosome” also refers to protein complexes containing exoribonucleases that are involved in mRNA degradation and the processing of small nucleolar RNAs (snoRNAs), small nuclear RNAs (snRNAs) and ribosomal RNAs (rRNA). Such protein complexes do not have membranes and are not “microvesicles” or “exosomes.” and thus are not EVs, as those terms are used here in.

As used herein, the term “patient” and “subject” are used interchangeably to refer to a human or animal, such as a vertebrate, e.g., a mammal. In some embodiments of the aspects described herein, the subject is a mammal, e.g., a primate, e.g., a human. A subject can be male or female. Additionally, a subject can be any stage of development, e.g., embryo, fetus, infant, child, pre-adolescent, adolescent, young adult, mature adult, and elderly adult. The female subject can be pregnant or not.

In some embodiments, the subject can be a patient or a subject in a clinical setting. The subject can be suspected of, or at risk for, having or developing a disease or disorder, or may have already been diagnosed as having a disease or disorder. The subject may be a patient undergoing treatment.

As used herein, a “capture agent” refers to any agent having specific binding for EVs generally (e.g., an exosome) or target EVs. Binding may be to a marker, e.g., a biomarker, which is present on all EVs, or to a subset of target EVs. Typically, the capture agent specifically binds to a biomarker fully or partially present on the external surface of the EVs (referred herein as an extravesicular marker), although in some embodiments, the capture agent specifically binds to a marker that is present on the interior of the EV (referred herein as an intravesicular marker). The capture agent is immobilized on the surface of a plasmonic nanostructure that is contacted to the sample (e.g., the sensing area). Examples of capture agents include, without limitation, nucleic acids, oligonucleotides, peptides, polypeptides, aptamers, antigens, polyclonal antibodies, monoclonal antibodies, single chain antibodies (scFv), antibody portions, F(ab) fragments, F(ab′)₂ fragments, Fv fragments, small organic molecules, polymers, compounds from a combinatorial chemical library, inorganic molecule, or any combination thereof.

A “nucleic acid,” as described herein, can be RNA or DNA, and can be single or double stranded, and can be, for example, a nucleic acid encoding a protein of interest, a polynucleotide, an oligonucleotide, a nucleic acid analogue, for example, peptide-nucleic acid (PNA), pseudo-complementary PNA (pc-PNA), locked nucleic acid (LNA) etc. Nucleic acid sequences include, for example, but are not limited to, nucleic acid sequences that act as transcriptional repressors, antisense molecules, ribozymes, small inhibitory nucleic acid sequences, for example, but not limited to, RNAi, shRNAi, siRNA, micro RNAi (mRNAi), antisense oligonucleotides etc.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or noncovalently associated therewith is still considered a “polypeptide.” Examples of modifications include glycosylation and palmitoylation. Polypeptides can be purified from natural sources, produced using recombinant DNA technology, synthesized through chemical means such as conventional solid phase peptide synthesis, etc. An “antigen” is defined herein as a substance inducing an immune response. The antigenic determinant group is termed an epitope, and the epitope in the context of a carrier molecule (that can optionally be part of the same molecule, for example, botulism neurotoxin A, a single molecule, has three different epitopes. Usually, antigens are foreign to the animal in which they produce immune reactions.

As used herein. “antibodies” can include polyclonal and monoclonal antibodies and antigen-binding derivatives, or portions or fragments thereof. Well-known antigen binding fragments include, for example, single domain antibodies (dAbs; which consist essentially of single VL or VH antibody domains), Fv fragment, including single chain Fv fragment (scFv), Fab fragment, and F(ab′)₂ fragment. Methods for the construction of such antibody molecules are well known in the art. As used herein, the term “antibody” refers to an intact immunoglobulin or to a monoclonal or polyclonal antigen-binding fragment with the Fc (crystallizable fragment) region or FcRn binding fragment of the Fc region. Antigen-binding fragments can be produced by recombinant DNA techniques or by enzymatic or chemical cleavage of intact antibodies. “Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The terms Fab, Fc, pFc′, F(ab′) 2 and Fv are employed with standard immunological meanings (see, e.g., Klein. Immunology (John Wiley, New York, N.Y., 1982); Clark, W. R. (1986) The Experimental Foundations of Modern Immunology (Wiley & Sons, Inc., New York); and Roitt, I. (1991) Essential Immunology, 7th Ed., (Blackwell Scientific Publications, Oxford).

The term “reporter group,” as used herein, refers to a composition capable of producing or enhancing a detectable optical signal indicative of the presence of a target in a sample. Examples of reporter groups include fluorescent molecules, such as fluorescein isothiocyanate (FITC), tetramethylrhodamine (TRITC), Alexa Fluor® family fluorophores such as AF405, AF488, AF555, and AF647, Cy3, Cy5, Cy5.5, DL755 and Cy7; small molecules for Raman signals, such as benzenethiol, 4,4′-bipyridine, and R6G; and nanoparticles made of metal, e.g., gold, semiconductor, plastic, polymer, and glass.

The term “label,” as used herein, refers to a composition capable of producing or enhancing a detectable signal indicative of the presence of the target in a sample.

As used herein, the term “marker” or “biomarker” refers to a molecule that is associated with an EV and can bind to a capture agent for detecting the EV. A marker can be any components of an EV that can be recognized by a capture agent. Examples of markers include, without limitation, proteins, or nucleic acids or a component of the lipid bilayer that makes up the membrane of the EV. Useful markers include receptors (e.g., extracellular) and channel components. A marker can be either an extravesicular or an intravesicular marker, as defined herein. A marker can be present on all EVs in a sample, or on a subset of EVs in a sample. A marker that is common to all EVs in a sample is referred to herein as a pan-EV marker.

As used herein, when one element is “fixed” to another, the two elements are directly connected via bonds, e.g., ionic, covalent, polar, or hydrogen bonds. When two elements are “bound” to each other, the two elements are directly or indirectly connected, via bonds, or via other elements, such as linker groups, e.g., PEG, or affinity ligands as described herein.

An “affinity ligand” is defined herein as a molecule that is directly attached or fixed to a molecular spacer or to a substrate or nanostructure, and also can be directly attached to a capture agent. Stated another way, an affinity ligand physically links a molecular spacer (or substrate or nanostructure) and a capture agent (or molecular spacer) together. In some embodiments, the affinity ligand is a first member of a specific binding pair. In such an embodiment, the capture agents may be the second member of the specific binding pair. Examples of such specific binding pairs include, without limitation, antigens, antibodies, haptens, oligonucleotides, polynucleotides, avidin, streptavidin, hormones, receptors, lectins, carbohydrates, IgG, protein A, and nucleic acid binding proteins. An affinity ligand can include, but is not limited to, a nucleic acid, oligonucleotide, peptide, polypeptide, antigen, polyclonal antibody, monoclonal antibody, single chain antibody (scFv), an antibody portion, F(ab) fragment, F(ab′)₂ fragment, Fv fragment, small organic molecule, polymer, compounds from a combinatorial chemical library, inorganic molecule, or any combination thereof.

Examples of specific binding pairs include antigen-antibody, hapten-antibody, or antibody-antibody pairs, complementary oligonucleotides or polynucleotides, avidin-biotin, streptavidin-biotin, hormone-receptor, ligand-receptors, lectin-carbohydrate, IgG-protein A, nucleic acid-nucleic acid binding protein, and nucleic acid-anti-nucleic acid antibody.

As used herein, the term “specific binding” refers to a chemical interaction between two molecules, compounds, cells, and/or particles wherein the first entity binds to the second, target entity with greater specificity and affinity than it binds to a third entity which is a non-target. In some embodiments, specific binding can refer to an affinity of the first entity for the second target entity that is at least 10 times greater than the affinity for the non-target entity. A reagent specific for a given target is one that exhibits specific binding for that target under the conditions of the assay being utilized. In some embodiments, specific binding is indicated by a dissociation constant on the order of ≤10⁻⁸ M, ≤10⁻⁹ M, ≤10⁻¹⁰ M or below.

Polyethylene glycol (PEG) is referred to herein as a possible component of the nano-plasmonic array and is used as a molecular spacer. A variety of forms and combinations of PEG are envisioned for use as such spacers. Polyethylene glycol (PEG) is a polyether compound with many applications from industrial manufacturing to medicine. The structure of PEG is (note the repeated element in parentheses): H—(O—CH₂—CH₂)ₙ—OH. PEG is also known as polyethylene oxide (PEO) or polyoxyethylene (POE), depending on its molecular weight. PEG, PEO, or POE refers to an oligomer or polymer of ethylene oxide. The three names are chemically synonymous, but as used herein, PEG refers to oligomers and polymers with a molecular mass below 20,000 g/mol, PEO refers to polymers with a molecular mass above 20.000 g/mol, and POE refers to a polymer of any molecular mass. PEG and PEO are liquids or low-melting solids, depending on their molecular weights. Different forms of PEG are also available, depending on the initiator used for the polymerization process—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete. Branched PEGs have three to ten PEG chains emanating from a central core group. Star PEGs have 10 to 100 PEG chains emanating from a central core group. Comb PEGs have multiple PEG chains normally grafted onto a polymer backbone. As used herein, “expression level” refers to the number of mRNA molecules and/or polypeptide molecules encoded by a gene of interest that are present in a cell or sample.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic illustration of a nanohole for comparison with the nanowells described herein.

FIG. 1B is a schematic illustration of an example of a nanowell described herein.

FIG. 1C is a schematic illustration of a method of making a nanowell described herein.

FIGS. 1D-1E are images and graphs that show the comparison of background noise of nanoparticles on nano pillars (NPOP) vs. nanowells (FIG. 1D), and the coefficients of variations (CVs)(FIG. 1E) of nanoholes and nanowells.

FIGS. 2A-2G are microscope images, schematics, and graphs that show the technical features of the fluorescence-amplified extracellular vesicle (FLEX) sensing technology.

FIGS. 3A-3D are microscope images and graphs that show the characterization of plasmon-enhanced EV detection with FLEX.

FIG. 4A is a graph that shows the reflectance spectrum of a nanowell array.

FIG. 4B is a pair of schematic diagrams and microscope images that show a comparison between conventional EV fluorescence and FLEX imaging.

FIGS. 5A-5F are a microscope image and graphs that show single CCA EV analysis with FLEX.

FIG. 6 is a heatmap graph that shows cell flow cytometry data.

FIGS. 7A-7B microscope images of tissue that show the result of patient tissue immunohistochemistry (IHC) studies.

FIGS. 8A-8D are heat map graphs and graphs that show EV protein profiling with FLEX.

FIGS. 9A-9E are schematic diagrams, graphs, and heatmaps that show the profiling of CCA patient EVs with FLEX.

FIGS. 10A-10B are bar graphs that show the proteomic analysis on cells and EVs.

FIG. 11 is a graph that shows the numbers of CD63-positive EV counts detected with FLEX and plain Au substrates.

FIG. 12 is a bar graph that shows the colocalization percentile between EV and marker channels.

FIGS. 13A-13B are bar graphs that show the marker positive rate with different threshold values.

FIG. 14 is a pair of bar graphs that show the repeatability and reproducibility tests for the FLEX assay.

FIGS. 15A-15D are a series of graphs that show the comparison of three bile-derived EV isolation methods.

FIG. 16 is a bar graph that shows the detected EV counts in bile samples. The error bar shows the standard deviation from 6 repeated measurements. The difference between groups was not statistically significant (p=0.34, Mann-Whitney test).

FIGS. 17A-17C are a series of graphs and a table (17C) that show the comparison of marker-positive EV counts and intensities between cholangiocarcinoma (CCA) and benign patient groups.

FIGS. 18A-18C are a series of graphs that show characterization of CCA data.

FIGS. 19A-19D are a series of microscope images and graphs, and a table (19D) that show EV characterization derived from biliary tract cancer (BTC) cell lines.

FIG. 20 is a graph and a series of images that show a comparison of fluorescence intensity values of 100-nm polystyrene beads using the 3×3 pixel intensity and dynamic window methods.

FIGS. 21A-21E are a pair of microscope images and a series of graphs that show the result of multi-channel fluorescence imaging of 100 nm multi-fluorescence beads on glass and FLEX substrates (Invitrogen, TetraSpeck).

FIGS. 22A-22C are a series of microscope images and a pair of graphs that show the optimization of the tetrafluorophenyl (TFP) labeling.

### DETAILED DESCRIPTION

The present disclosure relates to devices (e.g., nano-plasmonic arrays carrying a plurality of nanowells) and methods for analyzing extracellular vesicles (EVs) in a biological sample. The present disclosure is also related to methods of detecting a target analyte, e.g., a biomarker of a specific disease or disorder (e.g., cancer such as cholangiocarcinoma (CCA)), by analyzing the EVs from a sample (e.g., a liquid sample), e.g., obtained from a subject or the environment.

### Nanowell Nano-Plasmonic Chips

In one aspect, provided herein are nanowell nano-plasmonic chips for detecting target extracellular vesicles (EVs), the chip comprising, a substrate: a plurality of nanowells arranged to form an array (e.g., a periodic array or an aperiodic array) of nanowells on the substrate, wherein the array of nanowells is arranged and dimensioned to amplify one or more optical signals of electromagnetic radiation emitted, scattered, or reflected by EVs bound to the nanowells and/or EVs bound to the substrate near the nanowells, or to amplify one or more optical signals of electromagnetic radiation emitted, scattered, or reflected by reporter groups attached to the EVs; and one or more affinity ligands fixed on or adjacent to the nanowells, wherein the affinity ligands selectively bind to target EVs to bind the target EVs to the nanowells or to the substrate adjacent to the nanowells.

In particular, the present disclosure provides an improved form of a nano-plasmonic sensing technology, termed nanowell FLEX (fluorescence-amplified extracellular vesicle sensing technology), for sensitive and robust single EV analysis is disclosed. In the nanowell FLEX assay, EVs are captured on a plasmonic gold nanowell surface and immunolabeled for cancer-associated biomarkers to identify EVs, e.g., tumor-derived EVs (tEVs). The underlying plasmonic gold nanowell structures then amplify fluorescent signals of any EVs in a liquid sample, an effective amplification process at the single EV level. The nanowell FLEX EV analysis as described herein revealed a wide heterogeneity of tEVs and their marker levels. FLEX also detected small tEVs not detected by conventional EV fluorescence imaging due to weak signals.

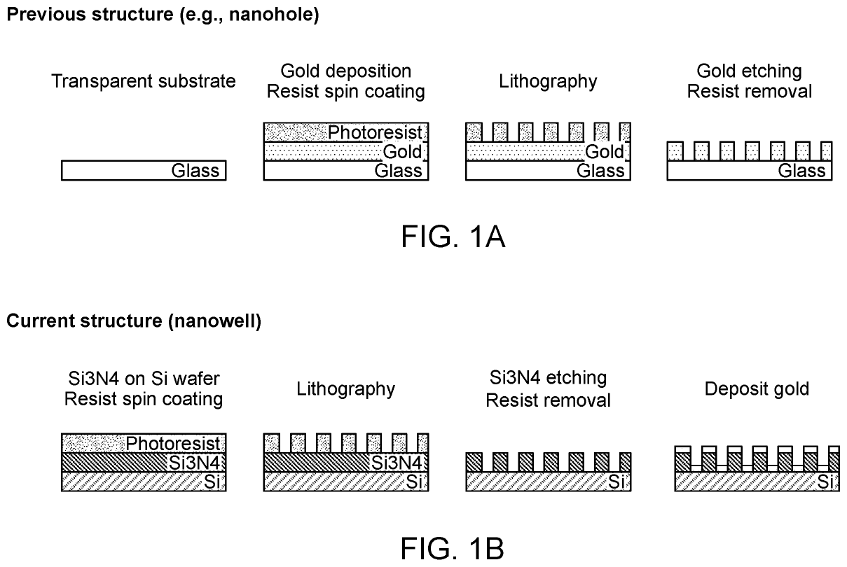

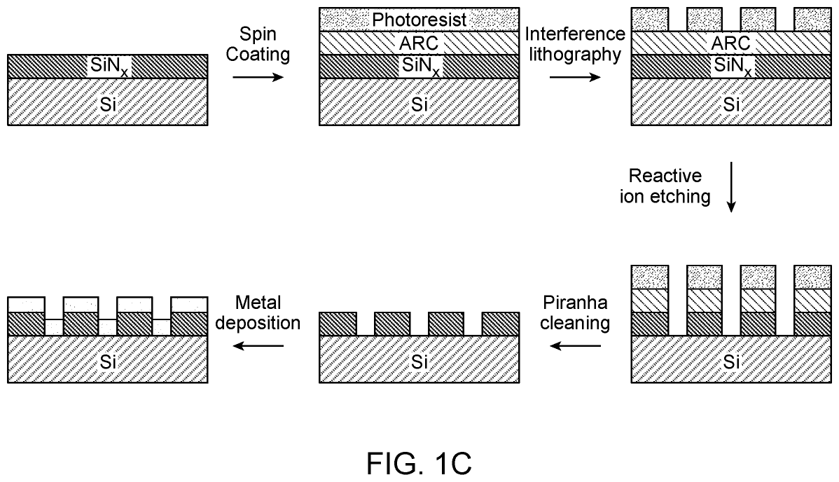

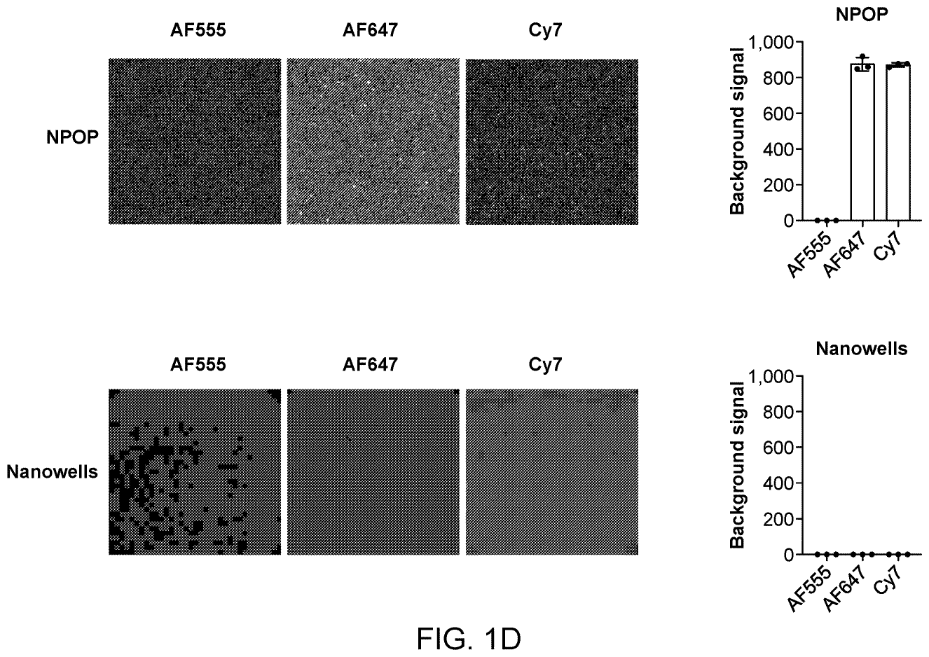

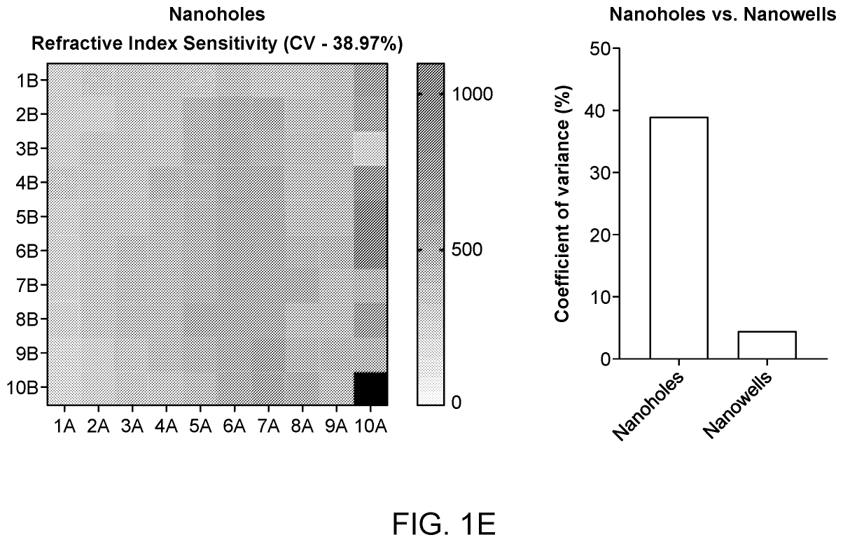

FIG. 1A is a schematic illustration of a previously disclosed nanohole chip for comparison with the presently described nanowell chip. FIG. 1B is a schematic illustration of an example of a nanowell chip described herein.

In general, the nanowells are coated with a layer of metal, e.g., gold (Au), silver (Ag), copper (Cu), aluminum (Al), platinum (Pt), or their alloys. In some embodiments, metal bilayers (e.g., Ag/Au bilayers) are used to coat the nanowells. The nanowells described herein provide a clean metal surface, which is essential for biosensing applications. In the previously disclosed nanostructures (e.g., the nanoholes), the gold surface needs to be covered by a photoresist layer and further exposed to chemicals during etching and resist removal. One also needs to dice the chip coated with the metal, which is another potential contamination source. However, in the nanowell chips described herein, the metal, e.g., gold, deposition is the last step, and no further processing is required. This simple process provides more precisely formed chips and better chip reproducibility within a batch and batch-to-batch.

Furthermore, the metal, e.g., gold, in the bottom of the nanowell can provide additional enhancement for EVs captured inside of the nanowells. For the previous nanohole nanostructures, where there is no metal inside the holes, and so there is less surface area for capturing the EVs.

In prior nanostructures (e.g., nanoholes), it was hard to adjust the thickness of metal on the nanostructures, because one needs to etch through the metal film. Etching a metal coating, such as a gold coating, is technically much more challenging than etching a layer of, for example, Si or Si₃N₄. In that regard, the presently described nanowell chips have far greater flexibility in terms of thicknesses and manufacturability.

In some embodiments, the thickness of the layer of metal, e.g., gold (Au), in the nanowell chips is about 10 nm-500 nm, e.g., about 20 nm-500 nm, about 30 nm-500 nm, about 40 nm-500 nm, about 50 nm-500 nm, about 100 nm-500 nm, about 150 nm-500 nm, about 200 nm-500 nm, about 250 nm-500 nm, about 300 nm-500 nm, about 350 nm-500 nm, about 400 nm-500 nm, about 450 nm-500 nm, about 10 nm-250 nm, about 20 nm-200 nm, about 30 nm-150 nm, about 40 nm-125 nm, about 50 nm-100 nm, about 100 nm-200 nm, or about 150 nm-200 nm. In some embodiments, the thickness of the metal layer (e.g., Au layer) is about 200 nm.

Any suitable non-metallic material can be used as the substrate of the devices described herein, including Si, SiO₂, Si₃N₄, polymers, and plastics that can support the nanostructures. In some embodiments, semiconductor wafers, e.g., silicon (Si) wafers, can be used as the substrate to manufacture the devices described herein, as the material is compatible with existing semiconductor device processes. These are also cheaper to manufacture and process than glass or quartz wafers. In some embodiments, the substrate is or includes silicon (Si), SiO₂, or Si₃N₄. In some embodiments, the substrate includes a patterned layer (also referred to as an etched layer or grooved pattern layer) that includes a lower layer of silicon (Si) and a surface or top layer of SiNₓ on top of the Si layer. In some embodiments, the patterned layer includes a surface layer of a non-metallic structure. In some embodiments, the patterned layer includes a non-metallic surface layer of SiO₂, Al₂O₃ or Hafnium oxide (HfO₂).

In some embodiments, the thickness of the lower layer of a patterned layer of the substrate (e.g., Si) is a few hundred microns, e.g., 100-1000 microns, 200-900 microns, 300-800 microns, 400-700 microns, or 500-600 microns, or a thickness of about 30 μm, about 50 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1000 μm. The thickness can be within the following ranges, about 50 μm-500 μm, about 100 μm-500 μm, about 150 μm-500 μm, about 200 μm-500 μm, about 250 μm-500 μm, about 300 μm-500 μm, about 350 μm-500 μm, about 400 μm-500 μm, or about 450 μm-500 μm, or about 100 μm-300 μm.

In some embodiments, the thickness of the patterned surface layer on the substrate (e.g., SiNₓ) is about 30 nm-1000 nm, 50 nm-900 nm, about 100 nm-800 nm, about 150 nm-700 nm, about 200 nm-600 nm, about 250 nm-550 nm, or about 300 nm-400 nm, or about 30 nm, about 40 nm, about 50 nm, about 100 nm, about 150 nm, about 200 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, or about 1000 nm. In some embodiments, the thickness of the substrate is about 100-200 nm.

In some embodiments, the nanowell has a 3D-structure with a cubic shape. In some embodiments, the nanowell has a 3D-structure with a cylindrical shape. In some embodiments, the nanowell has a diameter of about 50 nm-500 nm, about 100 nm-450 nm, about 150 nm-400 nm, about 200 nm-350 nm, about 250 nm-300 nm, or about 50 nm, about 60 nm, about 70 nm, about 80 nm, about 90 nm, about 100 nm, about 200 nm, about 300 nm, about 400 nm, or about 500 nm.

In general, the array of nanowells is a periodic array. The periodic arrangements support long-range surface plasmon resonance (induced by the periodic range) coupled with short-range SPR (induced by structure). Any suitable periodicity of the array of nanowells can be used in the nanowell nanoplasmonic chip described herein. In some embodiments, the periodicity of the nanowells provides a distance between the nanowells of about 100 nm-1000 nm, 200 nm-1000 nm, 300 nm-1000 nm, 400 nm-1000 nm, 500 nm-1000 nm, 600 nm-1000 nm, 700 nm-1000 nm, 800 nm-1000 nm, 900 nm-1000 nm, 100 nm-500 nm, 200 nm-500 nm, 300 nm-500 nm, 400 nm-500 nm. In some embodiments, the periodicity of the nanowells is a distance between the nanowells of about 500 nm.

Aperiodic arrays (e.g., random placement of nanostructures on a substrate) can also be used for plasmon-enhanced EV sensing. For example, nanoparticles on nanopillars (NPOP) can be manufactured with a random placement of nanostructures on a substrate.

The nano-plasmonic chips described herein can include any suitable number of nanowells, e.g., about 10 to about 100, about 10 to about 10³, about 10 to about 10⁴, about 10 to about 10⁵, about 10 to about 10⁶, about 10⁰ to about 10³, about 100 to about 10⁴, about 100 to about 10⁵, about 10⁰ to about 10⁶, about 10³ to about 10⁴, about 10³ to about 10⁵, about 10³ to about 10⁶, about 10⁴ to about 10⁵, about 10⁴ to about 10⁶, about 10⁵ to about 10⁶ or more, nanowells.

In some embodiments, the depth of the nanowells is about 50 nm-1000 nm, about 100 nm-1000 nm, about 200 nm-1000 nm, about 300 nm-1000 nm, about 400 nm-1000 nm, about 500 nm-1000 nm, about 600 nm-1000 nm, about 700 nm-1000 nm, about 800 nm-1000 nm, about 900 nm-1000 nm, about 50 nm-500 nm, about 100 nm-500 nm, about 200 nm-500 nm, about 300 nm-500 nm, about 400 nm-500 nm.

Any suitable affinity ligands can be used in the nano-plasmonic chips described herein. In some embodiments, the affinity ligands comprise polyethylene glycol (PEG). In some embodiments, the plurality of affinity ligands is fixed on or adjacent to the nanowells, and the plurality of affinity ligands bind, e.g., specifically bind, to EVs or target EVs via a capture agent. Different types of affinity ligands can be used in the nano-plasmonic chips based on a corresponding EV preparation. For example, among high affinity binding pairs, the substrate of the nano-plasmonic chips can include a biotin-binding protein (e.g., avidin) as the affinity ligands attached on the substrate, then the EVs or target EVs are required to comprise a corresponding biotin as the capture agent to be captured by the nano-plasmonic chips.

Suitable optical signals, such as fluorescence, Raman, and/or dark-field signals, can be detected and/or measured in the analysis of the nano-plasmonic chips described herein, as known in the art. In some embodiments, the EV analysis is performed using reporter groups, such as one or multiple fluorescent labels, to detect target EVs for EV analysis. For fluorescence detection, EVs are labeled by fluorescence probes conjugated with affinity ligands. For Raman detection, molecules on the surface membrane or inside of EVs can be directly detected or EVs are labeled with Raman probes conjugated with the affinity ligands. For dark-field scattering detection, scattering signals from EVs can be directly detected without any labeling. The nanowells of the nano-plasmonic chips are labeled with affinity ligands that bind, e.g., specifically bind, to EVs or are bound to capture agents that specifically bind to EVs, and then the substrate is exposed to a biological sample for a sufficient time to ensure that the substrate is bound to a sufficient number of EVs.

Examples of reporter groups include fluorescent molecules, such as fluorescein isothiocyanate (FITC), tetramethylrhodamine (TRITC), Alexa Fluor® 488, Cy3, Cy5, Cy5.5, and Cy7; small molecules for Raman signals, such as benzenethiol, 4,4-bipyridine, and R6G; and nanoparticles made of metal, semiconductor, plastic, polymer, and glass. Another example of reporter groups are those bound to capture agents that bind to a triplet marker (Mucin 1, cell surface associated (MUC1), Epidermal Growth Factor Receptor (EGFR), epithelial cellular adhesion molecule (EpCAM)), which is particularly effective in diagnosing CAA.

### Methods of Producing Nanowell Nano-Plasmonic Chips

In one aspect, the present disclosure provides methods of producing nano-plasmonic chips. As shown in FIG. 1C, one example of such a method includes the steps of obtaining a substrate, e.g., in the form of a non-metallic material, such as a semiconductor material, e.g., a silicon (Si) wafer, with a non-metallic surface layer, e.g., of silicon nitride (SiNx); applying an anti-reflection coating (ARC) and a photoresist coating on top of the surface layer; patterning nanowell arrays or patterning sub-wavelength wells (e.g., nanowells having a diameter smaller than 400 nm), e.g., using wafer-scale lithography methods, such as DUV lithography, interference lithography, and nanoimprint lithography; depositing a layer of gold (Au) on the top of the patterned arrays, thereby producing a nano-plasmonic chip. In some embodiments, after the lithography, reactive ion etching or the like can be used to remove the ARC and surface layer from the bottoms of the nanowells, before piranha cleaning and metal deposition.

The ARC is generally used in conjunction with interference lithography, whereas no ARC is required when using DUV, where just a single layer of photoresist can be used. When use, the ARC can be deposited on top of or below the photoresist coating. When used on top of the photoresist, the ARC reduces reflection of incident irradiation at the air/photoresist interface. An ARC deposited between the substrate and the photoresist minimizes the intensity of reflection of irradiation from the substrate. Any suitable anti-reflection coating (ARC) can be used in the nano-plasmonic chips described herein. In some embodiments, the ARC is a single-layer ARC, a dielectric anti-reflection (AR) coating, a multi-layer ARC, an anti-reflection V-coating, or a 2V coating. The ARC can be, for example, spin-coated onto the substrate before the photoresist coating is applied, e.g., also by spin-coating. The ARC can be baked onto the substrate before the photoresist is applied.

Any suitable photoresist coating can be used in the nano-plasmonic chips described herein. A photoresist is a light-sensitive substance that is used to generate a structured covering on a substrate in a variety of procedures such as photolithography. They are made up of polymers, sensitizers, and solvents. Each element serves a specific purpose. When exposed to radiation, the polymer's structure undergoes a modification. The solvent enables the photoresist to be twisted and thin layers to form on the silicon wafer. Finally, the sensitizer, also known as an inhibitor, regulates the photochemical process in the polymer network. Two distinct photoresists are utilized in semiconductor fabrication during photolithography. They are termed positive and negative photoresists. In some embodiments, the photoresist is a positive photoresist. In some embodiments, the photoresist is a negative photoresist.

Any suitable substrate (e.g., a substrate including a pattered layer) described herein can be used in the methods described herein.

In some embodiments, the substrate is a Si wafer with a low-stress silicon nitride (SiNₓ) layer deposited by low-pressure chemical vapor deposition (LPCVD). In some embodiments, the Si wafer has a diameter or length of about 1, 2, 3, 4, 5, 6, 7, 8, 9, 10 or more inches. In some embodiments, the Si wafer is about 4 inches in diameter. The wafer can be from between about 250 microns to 925 microns or more, e.g., 275, 375, 525, 625, 675, 725, 775, or 925 microns thick.

In some embodiments, the SiNₓ layer is about 50 nm, 100 nm, 150 nm, 200 nm, 250 nm, 300 nm, 350 nm, 400 nm 450 nm, 500 nm, thick, or more. In some embodiments, the SiNₓ layer is about 200 nm thick.

After anti-reflection coating (ARC) and spin-coating of a photoresist (e.g., positive or negative photoresist), two images, e.g., orthogonal grating images, can be exposed to the photoresist to generate the nanowell patterns. Subsequent reactive ion etching, e.g., with carbon tetrafluoride (CF4), transfers the well patterns into the SiNx layer. The remaining resists are removed, e.g., by piranha cleaning. Finally, a layer of metal, e.g., gold (Au) is deposited on the top of the chip. In some embodiments, the metal layer, e.g., Au layer, includes a Ti adhesion layer.

Further details of producing nano-plasmonic chips can be found, e.g., in US Publication Nos. US20230160809A1 and US20230123746A1, the entire contents of which are hereby incorporated herein by reference.

### Methods of Detecting Cancer

In one aspect, the present disclosure provides methods of detecting cholangiocarcinoma (CCA) in a subject. The methods include obtaining a sample from the subject; isolating extracellular vesicles (EVs) from the sample; capturing EVs isolated from the sample on a plasmonic chip bearing an array of nanostructures coated with a layer of gold; and detecting the presence and/or levels of one or more of MUC1, EGFR and EpCAM; wherein a higher expression level of one or more of MUC1, EGFR and EpCAM compared to a corresponding reference level indicates that the subject has CCA.

Also provided herein are nano-plasmonic chips for use in detecting cholangiocarcinoma (CCA) in a subject, wherein the detection comprises: obtaining a sample from the subject; isolating extracellular vesicles (EVs) from the sample; capturing EVs isolated from the sample on a nano-plasmonic chip bearing an array plurality of nanostructures coated with a layer of gold; and detecting the presence and/or levels of one or more of MUC1, EGFR and EpCAM: wherein a higher expression level of one or more of MUC1, EGFR and EpCAM compared to a corresponding reference level indicates that the subject has CCA.

Cholangiocarcinoma (CCA) is a fatal malignancy with a 5-year survival rate below 20%^{[1]} Along with pancreatic cancer, they are the only two cancers with increasing incidence and mortality rates. The high mortality rate is attributed to CCA's aggressiveness, late diagnosis, and refractoriness to chemotherapy.^{[2]} When present, CCA most commonly manifests as biliary obstruction, but clinical hurdles remain for diagnosis with endoscopic retrograde cholangiopancreatography (ERCP) or percutaneous biliary drainages. With ERCP, it has been reported that up to 20% of brush biopsies are found inconclusive, requiring repeated procedures and delaying treatment.^{[3,4]} The only diagnostic biomarker currently recommended for CCA is carbohydrate antigen 19-9 (CA19-9), but it also has suboptimal sensitivity and specificity, ranging 70-80%.^{[5,6]} The CA19-9 level can be elevated even with benign biliary strictures, especially for patients with obstructive jaundice. Therefore. CA19-9 alone is insufficient to distinguish between malignant and benign cases.^{[6,7]}

Bile contains secretomes from CCAs, such as proteins,^{[8,9]} nucleic acids,^{[10,11]} cytokines,^{[12]} and extracellular vesicles (EVs).^{[13-15]}. More sensitive ways of bile analysis could provide unique opportunities to screen and identify new biomarkers, as the biofluid likely contains biomarkers shed from CCA cancer cells locally. Recent studies have shown that the molecular analysis of bile obtained from ERCP outperformed plasma analysis for CCA diagnosis.^{[10,16]} For instance, next-generation sequencing of cell-free DNAs in bile showed a higher detection sensitivity for CCA, but the specificity remained below 70%. In contradistinctions, EVs are more abundant and stable and have been found in various body fluids such as ascites,^{[17]} bronchoalveolar lavage fluid,^{[18]} urine,^{[19]} bile,^{[13-15]} among others.

EVs are membrane-bound nanovesicles actively shed by cells into circulation. Tumor-derived EVs (tEVs) carry proteins and RNAs reflective of originating tumor cells,^{[20]} and thus serve as surrogate tumor markers. However, EV biomarker studies for CCA are relatively limited compared to other cancer types. One study investigated EV concentrations in bile samples as a marker to discriminate patients with malignant from those with nonmalignant biliary stenosis.^{[11]} Because EVs are shed not only by tumor cells but also by host cells, EV counts alone can be non-specific and have shown lower diagnostic powers in large cohort studies.^{[21,29,35,36]} Therefore, detecting tEVs based on their molecular profiling of tumor biomarkers is critical.

Recent studies support single EV analysis technologies as the most promising option for early cancer detection^{[22-33,37,38]}. This is because almost all types of cells shed EVs as background: tEV amounts can be minuscule in small sizes of primary tumors; and not all tEVs contain tumor biomarkers. Single EV detection can improve our understanding of EVs' various subtypes and heterogeneity and enables quantitative analysis^{[39-44]}. A key component of single EV analysis remains robust signal amplification due to EVs' weak signals associated with limited surface areas and epitopes available for immunolabeling. Among various signal amplification strategies, such as branched DNA probes^{[47]} or enzymatic reactions,^{[22]} plasmon-enhanced fluorescence has shown robust fluorescence signal amplification across multiple channels.^{[48,49]} Methods of EV detection using plasmon-enhanced fluorescence imaging have been described in detail in, e.g., US Publication Nos. US20230160809A1 and US20230123746A1, the entire contents of which are incorporated herein by reference.

Here, the development of an advanced plasmonic EV analysis technology for sensitive and robust single EV analysis is reported. Termed “FLEX” (fluorescence-amplified extracellular vesicle sensing technology), the technology harnesses plasmon-enhanced fluorescence detection of EVs captured on gold nanowell structures (e.g., one or more periodic arrays of nanowells), enabling EV protein profiling at the single EV level using clinical samples. FLEX technology was applied to EV analyses in clinical bile samples from patients who underwent ERCP.

Any other suitable nanostructures can be used in the methods described herein. For example, nanoholes, nanorods, nanodisks, nanowells, nanosquares, nanopillars, or nanogrooves can be used in the methods described herein. The EVs can be captured either by covalent bonding/binding or by affinity ligands. For the former, the surface of plasmonic substrates (made of gold, silver, copper, aluminum, platinum, or their combination) is functionalized with a linker (e.g., PEG or carbon chains with functional groups) or biomolecule adhesion layer to capture all EVs on the surface via non-specific interactions. For the latter, marker-positive, target EV subpopulations are captured on the surface via affinity ligands/capture agents (e.g., antibodies, aptamers, peptides, nucleic acids). The first case is the capture of all EVs. The second case is the capture of target EVs. Suitable nanostructures are described in, e.g., US Publication Nos. US20230160809A1 and US20230123746A1, the entire contents of which are hereby incorporated by reference.

The captured EVs can then be immune-labeled for marker analysis. In some embodiments, the method comprises immunolabeling the captured EVs using a binding ligand. As used herein, a “binding ligand” is a ligand that specifically binds to the EVs. In some embodiments, the binding ligand is labeled with a reporter group. Any suitable reporter group known in the art can be used herein. For example, the binding ligand can be labeled with a fluorescent reporter group. The binding ligand can be labeled with a chemical reporter group.

Examples of reporter groups include fluorescent molecules, such as fluorescein isothiocyanate (FITC), tetramethylrhodamine (TRITC), Alexa Fluor® 488, Cy3, Cy5, Cy5.5, and Cy7; small molecules for Raman signals, such as benzenethiol, 4,4′-bipyridine, and R6G; and nanoparticles made of metal, e.g., gold, semiconductor, plastic, polymer, and glass. Another example of reporter groups is a QUAD marker (Mucin 1, cell surface associated (MUC1), Epidermal Growth Factor Receptor (EGFR), epithelial cellular adhesion molecule (EpCAM), and human epidermal growth factor receptor 2 (HER2)). In some embodiments, the reporter group is a fluorophore. In some embodiments, the fluorophore is selected from Cy3, Cy5, AF367, AF405, AF488, AF555, AF647, DL755, and Cy7.

In some embodiments, the binding ligand is selected from antibodies targeting surface or transmembrane proteins, proteins inside the EVs, and fluorophore-conjugated nucleic acid probes.

In some embodiments, the antibodies can be fluorophore-conjugated fluorescence primary antibodies or unconjugated primary antibodies with fluorophore-conjugated secondary antibodies labeling the primary antibody.

In some embodiments, when using antibodies targeting proteins inside the EVs, EV membrane permeabilization is carried out.

In some embodiments, the fluorophore-conjugated nucleic acid probes are selected from aptamers, molecular beacons, and DNA strands.

The methods described herein allow for improved sensitivity for the detection of EVs (e.g., in a liquid sample from a subject). In some embodiments, the methods described herein captures about or at least 10%, 15%, 20%, 25%, 30%, 35%, 40%, 45%, 50%, 55%, 60%, 65%, 70%, 75%, 80%, 85%, 90%, 95% 96%, 97%, 98%, 99% or 100% of the EVs in a sample (e.g., a bile sample from a subject).

In some embodiments, the methods described herein detects about or at least 10%, 15%, 20%, 25%, 30%, 35%, 40%, 45%, 50%, 55%, 60%, 65%, 70%, 75%, 80%, 85%, 90%, 95% 96%, 97%, 98%, 99% or 100% of the EVs in a sample (e.g., a bile sample from a subject).

In some embodiments, the methods described herein detects a cancer (e.g. CCA) by the detection of about or at least 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰ EVs in a sample (e.g., a bile sample from a subject).

In some embodiments, an EV can be detected by a single protein labeled on the EV, e.g., using the signal of a reporter group. In some embodiments, an EV can be detected by about or at least 1, 2, 3, 4, 5, 6, 7, 8, 9, 10, 10², 10³, 10⁴, 10⁵ proteins labeled on the EV, e.g., using the signal of a reporter group.

In some embodiments, one or more of MUC1, EGFR and EpCAM is detected for the detection of CCA. In some embodiments, MUC1 is detected for the detection of CCA. In some embodiments, EGFR is detected for the detection of CCA. In some embodiments, EpCAM is detected for the detection of CCA. In some embodiments, MUC1 and EGFR are detected for the detection of CCA. In some embodiments, MUC1 and EpCAM are detected for the detection of CCA. In some embodiments, EpCAM and EGFR are detected for the detection of CCA. In some embodiments, MUC1. EGFR and EpCAM are detected for the detection of CCA.

In some embodiments, the presence and/or levels of one or more of MUC1. EGFR and EpCAM is determined by the fluorescence signal of the binding ligands.

Any other suitable markers for the detection of cancer (e.g., CCA) can be used in the methods described herein. Cancer marker are generally known in the art. For example, the National Cancer Institute website (Tumor Markers—NCI (cancer.gov)) lists common cancer markers.

In some embodiments, the method further comprises imaging the plasmonic chip by plasmon-enhanced multi-channel imaging. The present disclosure provides improved multi-channel imaging methods for the detection of cancer (e.g., CCA).

In some embodiments, the plasmon-enhanced multi-channel imaging comprises amplifying fluorescent signals from the plasmonic gold nanowell arrays at multiple wavelengths to improve single EV detection sensitivity.

In some embodiments, the plasmon-enhanced multi-channel imaging comprises co-localization analysis from multiple imaging channels and co-existing marker analysis on a single EV level.

In some embodiments, the corresponding reference level is a level of expression of MUC1, EGFR, and/or EpCAM on EVs from a healthy subject.

Any suitable nanostructures can be used in the methods described herein. In some embodiments, the nanostructures are nanowells, nanoholes or nanopillars. In some embodiments, the nanostructures are nanowells described herein.

Any suitable sample can be used in the methods described herein. In some embodiments, the sample is a liquid biological sample, In some embodiments, the sample is bile.

The size of EV can be any suitable size known in the art. In some embodiments, the EVs have a diameter of about 10-1000 nm, about 50-1000 nm, about 100-1000 nm, about 500-1000 nm, 10-500 nm, about 50-500 nm, about 100-500 nm. In some embodiments, the EVs have a diameter of about 50-200 nm.

Further details of using nano-plasmonic chips to capture and detect EV-associated markers can be found, e.g., in US Publication Nos. US20230160809A1 and US20230123746A1, the entire contents of which are hereby incorporated herein by reference.

### EXAMPLES

The invention is further described in the following examples, which do not limit the scope of the invention described in the claims.

### Materials and Methods

The following materials and methods were used in the following examples.

### Clinical Sample Collection

Bile samples were prospectively collected at the Severance Hospital, Yonsei University College of Medicine, Seoul, Korea, from 26 patients undergoing ERCP due to biliary obstruction. One bile sample was excluded from the analysis due to a large amount of impurities and large aggregates during EV isolation. Among the 25 samples analyzed, 17 patients were diagnosed with cholangiocarcinoma, and 8 patients were diagnosed with benign biliary obstruction. All ERCP procedures with therapeutic video duodenoscopy (TJF-260V) were performed by interventional endoscopists with experience in at least 1000 cases. Experienced attending anesthesiologists sedated all the patients. After selective biliary cannulation, 5-10 mL of bile was aspirated via a biliary catheter. After acquiring a cholangiogram to evaluate the biliary obstruction, tissue acquisition was performed by using intraductal biopsy if malignancy was suspected. Diagnosis of cancer was established by the following methods in all cases: (i) surgical pathology; (ii) pathologic diagnosis made by ERCP tissue biopsy with evidence for malignancy; and (iii) pathologic diagnosis made by other tissue acquisition methods, such as percutaneous biopsy or endoscopic biopsy for metastasis or direct invasion of the tumor to other organs. For patients with benign conditions, such as choledocholithiasis, we followed up the patients for at least 1 year without evidence of malignancy. The protocol of the present study adhered to the Declaration of Helsinki and was approved by the Institutional Review Board of Severance Hospital (IRB number: 4-2018-1115) and Massachusetts General Hospital. Written informed consent was obtained from all subjects.

EV Isolation from Bile Samples

1 mL of bile sample from each patient was centrifuged with 300×g for 10 min at 4 C to remove floating cells or large debris. Then, the bile sample was centrifuged with 2,000×g for 20 min at 4 C to remove larger particles, such as apoptotic bodies. We isolated EVs using 3 methods (ultracentrifugation (UC), size-exclusion chromatography (SEC), and a combination of UC and SEC) and compared the better methods. For EV isolation using UC, the sample was centrifuged 100,000×g for 70 min at 4° C. with a polypropylene tube for SW 32.1 Ti rotor of Optima™ Ultracentrifuge (Beckman Coulter). Then, the pellet was washed with PBS and centrifuged again with 100,000×g for 70 min at 4° C. The EV pellet was resuspended in PBS. For EV isolation using SEC, we first prepared an SEC column with Sepharose CL-4B (GEHealthcare) based on the previously published protocol.^{[66]}

Briefly, an 11 μm pore-sized nylon membrane (NY 1102500, Millipore Sigma) was placed on the bottom of a 10 mL syringe (BD Biosciences). The syringe was stacked with 10 mL of Sepharose and triple-washed with PBS. Then, the bile sample was applied, and the 4th and 5th fractions (1 fraction=1 mL) were collected for EVs. The collected sample was concentrated using Amicon Ultra-2 Centrifugal Filter (MWCO=10 kDa, Millipore Sigma) and centrifuged at 3,500×g for 30 min at 4° C. The isolated EVs were resuspended in PBS. For EV isolation using a combination of UC and SEC, EVs were first isolated by UC with 100,000×g for 70 min at 4° C. The pellet was diluted in 1 mL of PBS and passed through an SEC column. The 4th and 5th fractions were collected and centrifuged at 3,500×g for 30 min at 4° C. with an Amicon Ultra-2 Centrifugal Filter (MWCO=10 kDa, Millipore Sigma). The isolated EVs were resuspended in PBS. The isolated EVs were fluorescently labeled using the published protocol.^{[26]} Briefly, EVs were mixed with 0.2 μl TFP-AF555, followed by 1 hr of incubation. The labeled EV was filtered with a 40 K MWCO column (Thermo Fisher) to remove the remaining dye and was diluted in PBS before the assay.

### Cell Lines

SNU308, SNU478, and SNU1196 were provided by Yonsei University. SNU308, SNU478, and SNU1196 were grown in Roswell Park Memorial Institute (RPMI) 1640 medium (Gibco) supplemented with 10% fetal bovine serum (FBS. Thermo Fisher), 100 U/mL penicillin, and 100 μg/mL streptomycin (Gibco) at 37° C. in 5% CO2. H69 cells were generously provided by Prof Yangmi Kim and Prof Seon Mee Park at Chungbuk National University College of Medicine. H69 cells were maintained in enriched Dulbecco's minimum essential medium (DMEM) (Hyclone) containing 10% fetal bovine serum (FBS) (Gibco, Invitrogen), 0.025 mg/ml adenine (Sigma, St. Louis, Mo. USA), 0.005 mg/ml insulin (Gibco Invitrogen), 0.002 mg/ml epinephrine (sigma), 13.6 ng/ml T3T triiodo_L_thyronine (T3) (sigma), 0.0083 mg/ml holo-transferrin (Gibco, Invitrogen). 620 ng/ml hydrocortisone (Sigma) and 10 mg/ml epidermal growth factor (EGF; CytoLab Ltd., Rehovot, Israel) at 37° C. in 5% CO2. All cell lines were tested and free from mycoplasma contamination (Universal Mycoplasma Detection Kit, ATCC).

EV Isolation from Cell Lines

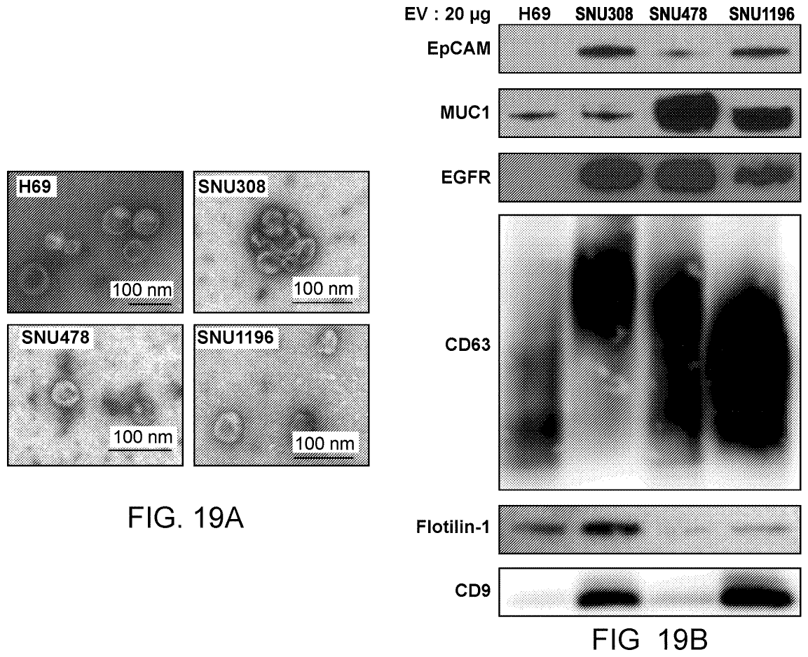

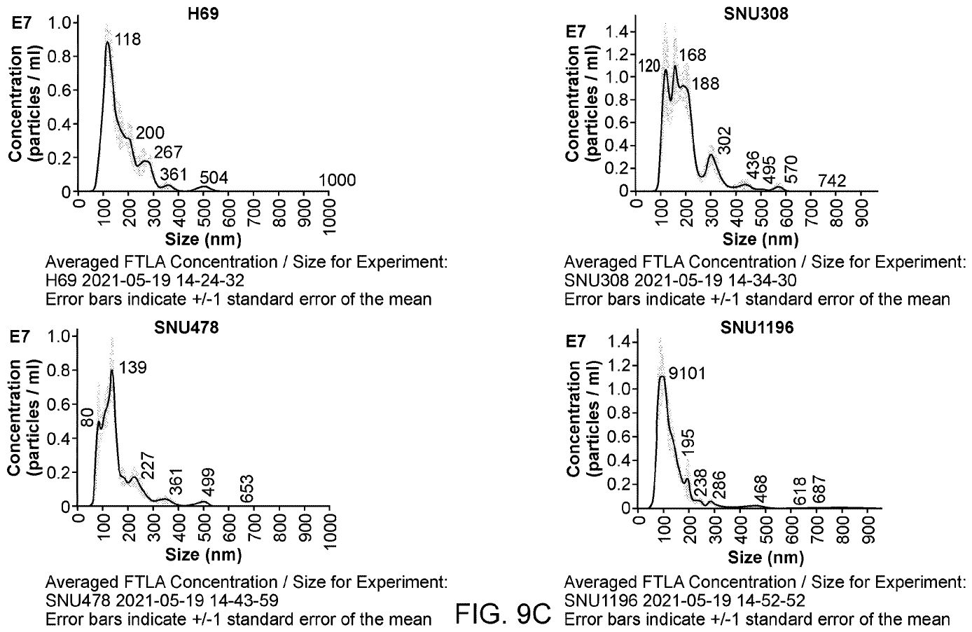

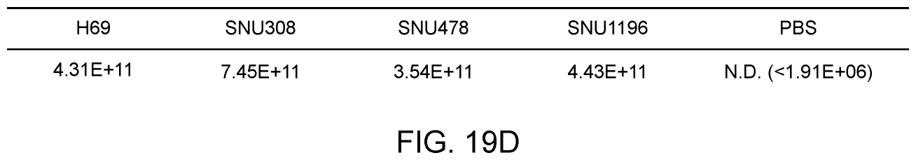

Cells were incubated in a medium with 2% exosome-depleted FBS (Thermo Fisher) for 48 h, followed by EV collection. The conditioned medium was collected through a cell strainer (40 μm Nylon, Thermo Fisher) and filtered through a 0.2 μm membrane filter (Millipore Sigma). The conditioned medium was concentrated with Centricon Plus-70 Centrifugal Filter (MWCO=10 kDa, Millipore Sigma) and centrifuged at 3,500 g for 30 min at 4° C. The concentrated medium was passed through with SEC. Similar to EV isolation from bile samples, the 4th and 5th fractions were used for EV isolation, followed by concentration using Amicon Ultra-2 Centrifugal Filter (MWCO=10 kDa, Millipore Sigma) and centrifuged at 3,500×g for 30 min at 4° C. The isolated EVs were reconstituted in PBS, aliquoted, and stored in a −80 C deep freezer. Total EV protein was measured using a Qubit assay kit (ThermoFisher, Q33212). The isolated EVs were characterized by transmission microscopy, western blot, and nanoparticle tracking analysis (FIGS. 19A-19D).

### FLEX Chip Fabrication

We first patterned periodic nanowell arrays (200 nm in diameter and 500 nm in periodicity) using interference lithography, which was done through LumArray, Inc (Somerville, MA, USA). Briefly, a 200-nm thick, low-stress silicon nitride layer was first deposited by low-pressure chemical vapor deposition (LPCVD) on 4-inch Si wafers. After anti-reflection coating (ARC) and spin-coating of a negative photoresist, two orthogonal grating images were exposed to the photoresist and made periodic nanowell patterns. Subsequent reactive ion etching with CF₄ transferred the hole patterns into the silicon nitride layer. The remaining resists were removed by piranha cleaning.^{[67]} Deposition of 100-nm thick Au with a 5-nm Ti adhesion layer produced periodic Au nanowell arrays. The wafers were then diced into smaller pieces for EV assays.

### FLEX EV Assay

FLEX chips were serially cleaned with acetone, IPA, and deionized water. The sensor chip surface is functionalized with a mixture of SH-PEG-COOH 1 k (Nanocs) and SH-mPEG 0.35 k at a ratio of 1:3 overnight. For EV capture, the gold surface was incubated in 0.2 M EDC (Thermo) and 0.05 M sulfo-NHS (Thermo) for 7 min to capture EVs by covalent bonding. After gently washing with PBS, EVs were introduced to the sensor chip and incubated for 30 min. After EV capture, EVs were fixed by 4% paraformaldehyde for 10 min, followed by blocking with 2% BSA for 20 min. The captured EV were immuno-fluorescently labeled by primary antibodies for 60 min, followed by secondary antibody (AlexaFluor 647 anti-mouse) incubation for 30 min. Antibodies were diluted in 0.2% BSA solution, and staining was performed under agitation. Each antibody was diluted with its dilution factor, which was determined by screening optimal antibody concentrations (CD63:1/100, EpCAM:1/20, MUC1:1/800, EGFR:1:20). Finally, the chips were mounted with a mounting solution (ProLong Gold Antifade mountant. Thermo Fisher) and covered with a glass coverslip. Fluorescence images were acquired on Nikon Ti inverted automated epifluorescence microscope with a 40× (NA=0.95) objective lens.

### Image Processing

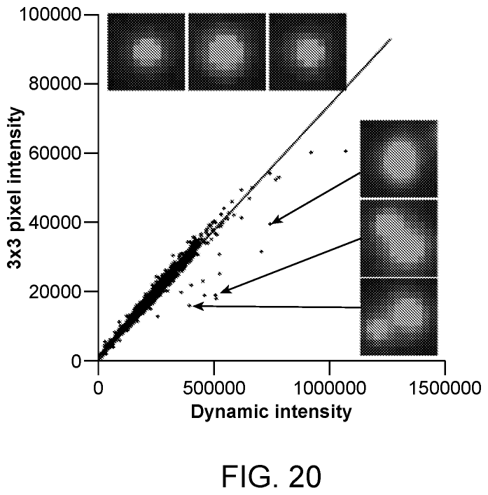

Images were analyzed using ImageJ (ImageJ2 Fiji, version 2.3.0/1.53q) and custom-built MATLAB (version R2015a) code. Image shift between fluorescence channels was registered and corrected using the ImageJ NanoJ plugin. We then subtracted background signals using a rolling ball algorithm (radius=50). We then use the ComDet plugin in ImageJ to detect EV locations using AF555 signals. Although the ComDet plugin also provides fluorescence intensities of individual detected particles from a dynamic pixel window, we found that the dynamic method sometimes overestimated intensity values when particles form dimers or larger aggregates (FIG. 20). Instead, we calculated averaged fluorescence intensities from a 3×3 fixed pixel window in AF647 channels.

### Flow Cytometry Analysis for Cell Lines

SNU308, SNU478, SNU1196, and H69 cells were trypsinized, washed with PBS twice, and then fixed with chilled 2% paraformaldehyde for 1 hour. Cells were blocked by 0.5% BSA and 2% normal fetal bovine serum on ice for 30 minutes. The cells were then incubated with primary antibodies and washed with PBS, followed by secondary antibody labeling with Alexa Fluor 488. The flow cytometry measurements were obtained with a BD FACS LSR II SORP system, and the data were analyzed using Flow Jo software (version 10.2). The excitation beam for the GFP was set at 488 nm, and the emission signal was captured with a 525/50 nm bandpass filter. The gain voltages were set by default to 625V, 420V, and 600V for FSC, SSC, and GFP acquisition, respectively, and events were created for measurements where FSC>200 & SSC>200.

### Flow Cytometry for EVs

10⁹-10¹⁰ EVs were incubated with 0.2 μL aldehyde/sulfate beads (Invitrogen, A37304) for 30 min to saturate the beads with EVs, followed by blocking with 1% BSA in PBS for 2 hrs. After coating with glycine, beads were washed with PBS using centrifugation. Then, beads were incubated with primary antibodies diluted to 10 μg/mL in PBS with 1% BSA for 1 hr, followed by another 1 h incubation with secondary antibodies. Samples were analyzed with a CytoFlex flow cytometer (Beckman Coulter, A00-1-1102) using the 488/8 and 525/40 nm bandpass filter for SSC and FITC, respectively, and the following settings (FSC 201V, SSC 90V, FITC159V). FlowJo X 10.0 was used for analyzing median fluorescence intensity.

For normalization, the median fluorescence intensity was divided with the signal of the isotope control. Moreover, the z-score was calculated using each marker's mean value and standard deviation.

### Transmission Electron Microscopy (TEM)

For sample preparation, we placed a drop of sample on the Formvar-carbon coated grid for 15 seconds, removed the droplet using filter paper, put a drop of 1% uranyl acetate for 15 seconds and removed it using filter paper, and washed it with a drop of distilled water. Dried grids were imaged with transmission electron microscopy (JEM-1011, JEOL, Tokyo, Japan) at the acceleration voltage of 80 kv equipped with a Megaview III CCD camera (Softimaging system—Germany).

### Immunohistochemistry

Immunohistochemistry (IHC) was done on paraffin-embedded tissue with Mucin 1 (MUC1), epithelial cell adhesion molecule (EpCAM), and epidermal growth factor receptor (EGFR) using the standard immunohistochemistry technique. IHC staining was done manually after antigen retrieval by boiling slides in 10 mM sodium citrate buffered distilled water (pH 6.0) for 20 minutes in a 97 C water bath, followed by a 30-minutes cooldown period. Primary antibodies used were monoclonal anti-MUC1 antibody (catalog no. 10-M93B) and anti-EpCAM antibody (catalog no. ab20160) at a dilution of 1:200 and anti-EGFR antibody (catalog no. sc365829) at a dilution of 1:100. Primary antibodies were incubated for 19 hours at 4 C. The Dako REAL Peroxidase Detection System Kit was used according to the manufacturer's instructions, including the ready-to-use-anti-rabbit/mouse secondary antibody (catalog no. K5007) and counterstained with hematoxylin solution (catalog no. 03971)

### Mass Spectrometry Analysis

EV samples were analyzed using an LC-MS/MS system consisting of an UltiMate 3000 RSLCnano system (Thermo Fisher Scientific) and an Orbitrap Eclipse Tribrid mass spectrometer (Thermo Fisher Scientific) equipped with a nano-electrospray source (EASY—Spray Sources, Thermo Fisher Scientific). Please see Supporting Information for sample preparation. Peptides from EV samples were trapped 75 μm×2 cm C18 pre-column (nanoViper, Acclaim PepMap100. Thermo Fisher Scientific) before being separated on an analytical C18 column (75 μm×50 cm PepMap RSLC, Thermo Fisher Scientific) at a flow rate of 250 nL/min and total run time of 70 min. Mobile phases A and B comprised 100% water containing 0.1% formic acid and 100% acetonitrile (ACN) containing 0.1% formic acid, respectively. The voltage applied to produce an electrospray was 2,000 V. During the chromatographic separation, the Orbitrap mass spectrometer was operated in data-dependent mode, automatically switching between MS1 and MS2. The MS data were acquired using the following parameters: Full scan MS1 spectral (400-2000 m/z) were obtained in the Orbitrap for a maximum ion injection time of 50 ms at a resolution of 120,000 and a standard mode automatic gain control (AGC) target. MS2 spectra were acquired in the Orbitrap mass analyzer at a resolution of 30.000 with turbo-TMT setting applying high energy collision dissociation (HCD) of 36% normalized collision energy and AGC target value of 5.0×10⁴ with a maximum ion injection time of 54 ms. Previously fragmented ions were excluded for 30 sec.

### Proteome Search and Bioinformatics Analysis

A proteome search was performed based on the previously published protocol^{[68]} with minor modifications as followed. The MS raw files were converted into mzML using MSConvert (version 3.0.20033). Ms2 files were extracted from the mzML using an in-house program coded by Python 3.8 from Anaconda 3 environment (version 3.8.0). To analyze the proteins in EVs, we generated a proteome database from Uniprot and Integrated Proteomics Pipeline version 5.1.2. (IP2, Integrated Proteomics Applications Inc., San Diego. CA). Proteome search results were evaluated by the false discovery rate (FDR) at spectra and protein level with less than 1.0% using DTASelect (Integrated Proteomics Applications Inc., San Diego, CA), respectively.

Protein quantification for the discovery of DEP was performed from the ms2 files with TMT reporter ions using the Census software (Integrated Proteomics Applications Inc., San Diego, CA). Each data was normalized with H69 data to show the fold change in cholangiocarcinoma cell lines and EVs over those in the H69 cell line and EVs.

### Nanoparticle Tracking Analysis

The number of EVs was measured by nanoparticle tracking analysis (Nanosight LM10 microscope. Malvern). The experiment was conducted with a 642 nm laser module at room temperature. Each sample was diluted 500-fold in PBS and manually placed in the chamber. Each experiment was performed for 30 seconds in quadruplicate. The number of particles per frame was 33.3-87.7, and the frame rate per second was 30.

### Statistical Analysis

The data were analyzed with GraphPad Prism version 9 (GraphPad Software Inc., San Diego, CA. USA). All data were displayed as mean f standard deviation. The Mann-Whitney unpaired t-test was used to compare two independent groups. For comparing more than two groups, we used a one-way ANOVA test. Statistical significance was accepted for values of p<0.05.

### Experimental Setup

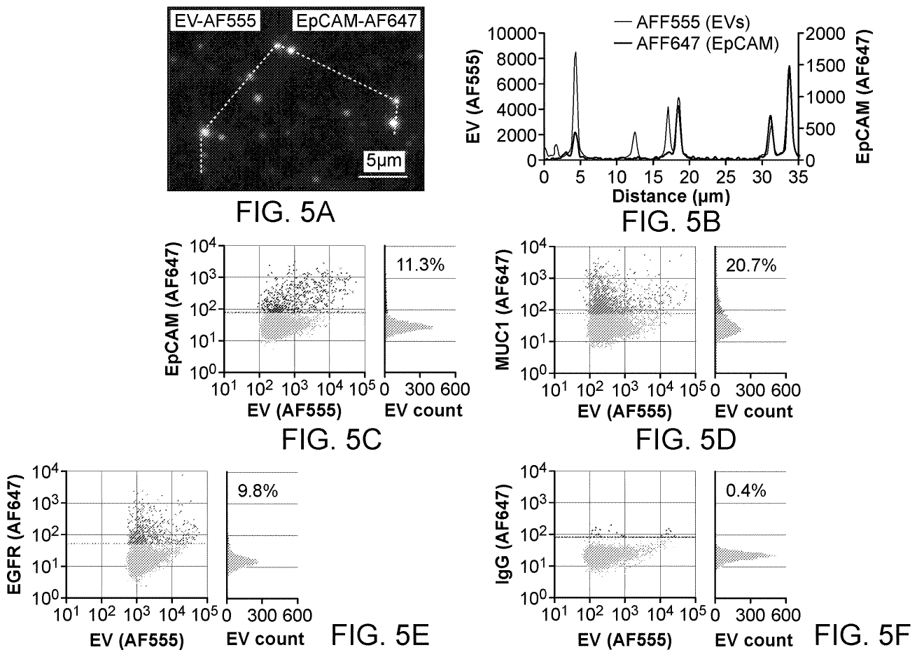

The FLEX chip characterization (FIGS. 5A-5F) was performed using an upright microscope (Zeiss. Axio Imager 2) which is equipped with a 40× NA 0.95 objective lens (Plan-Apochromat Corr D=0.25 M27) and a multicolor LED light source (Zeiss, Colibri 7 FR-R[G/Y]BV-UV). The images of the rest experiments were acquired using an inverted microscope (Nikon, Eclipse Ti), a 40× NA 0.95 objective lens (Nikon, MRD70470), and a mercury vapor lamp-based light source (Excelitas technologies. X-Cite 120Q).

### Spectrum Measurement

The reflection spectrum was measured by a Nikon 50i upright microscope using a 10× (N.A.=0.1) objective lens. A halogen light source was illuminated by Kohler illumination and reflected light was collected at a camera port using a convex lens (f=25 mm, Thorlabs). A multimode fiber was located at the focal point of the lens, and the signal was acquired by a USB spectrometer (Ocean Optics).

### Chip Characterization

1 wt % polyvinyl alcohol (PVA, MW. 13 000-23 000; Sigma-Aldrich) was prepared in water and mixed using a microwave oven. AlexaFluor 488, 555, and 647 dyes were diluted to 50 μM. The dye-containing PVA films were deposited by spin-coating at 3000 rpm, and the thickness is estimated at ~25 nm.

### Sample Preparation for Proteomics

Proteomic analysis, including LC-MS/MS, was performed at the Korea Basic Science Institute (Rep. of Korea. Chungbuk). Each sample was digested in an S-Trap mini spin column (Protifi, USA) according to the manufacturer's instructions. Extracted EVs were homogenized for 20 sec by 5% SDS in 50 mM TEAB. Briefly, 100 g of proteins was heated to 95° C. for 5 min, reduced with 5 mM TCEP (final concentration) for 1 hr at 60° C., and alkylated with iodoacetamide at a final concentration of 20 mM in the dark for 40 min. The alkylated proteins were acidified by adding phosphoric acid to a final concentration of 1.2% and mixed with six volumes of binding buffer (90% methanol; 100 mM TEAB; pH 7.1). After gentle mixing, the protein solution was loaded onto the filter and centrifuged at 4.000×g for 30 sec. Then the samples were washed two to three times with a 90:10 methanol:TEAB (50 mM) solution and digested with trypsin gold (Promega) at 37° C. for overnight at a protein-to-enzyme ratio of 10:1 (w/w). Peptides were eluted stepwise with three elution buffers at a volume of 200 μL each with one more repeat, including 50 mM TEAB in water, 0.2% formic acid in water, and 50% acetonitrile/0.2% formic acid in water.

### Tandem Mass Tag (TMT) Labeling and High pH RPLC Fractionation

Peptide samples (80 g each) were labeled with TMT reagent (Thermo Scientific, Rockford, IL) according to the manufacturer's instructions. Each TMT channel powder was freshly dissolved in anhydrous acetonitrile (ACN) with a ratio of 0.8:41 (w:v, mg:μL). After incubation for 1 hr at room temperature, the reaction was quenched by adding 8 μL of 5% hydroxylamine, and the labeled peptide samples were incubated for 15 min. 2.5% of TMT-labeled peptides from each channel were prepared for LC-MS/MS analysis for determining labeling efficiency before pooling. Pooled TMT-labeled peptides from 6 channels were desiccated by Speed-Vac for High-pH fractionation. A high pH reversed-phase peptide fractionation kit (Thermo Fisher Scientific) was used to fractionate TMT-labeled peptides by increasing acetonitrile step-gradient elution. First, the column was equilibrated with acetonitrile and 0.1% trifluoroacetic acid (TFA). Second, the mixed labeled peptide samples and pure water were loaded and desalted by low-speed centrifugation. Finally, the column was combined with high-pH acetonitrile solution with increasing concentration. The peptides were subjected to gradient elution, and each eluted peptide sample was vacuum dried.

### Example 1: Nanowell FLEX Technology for Plasmon-Enhanced Single EV Analysis

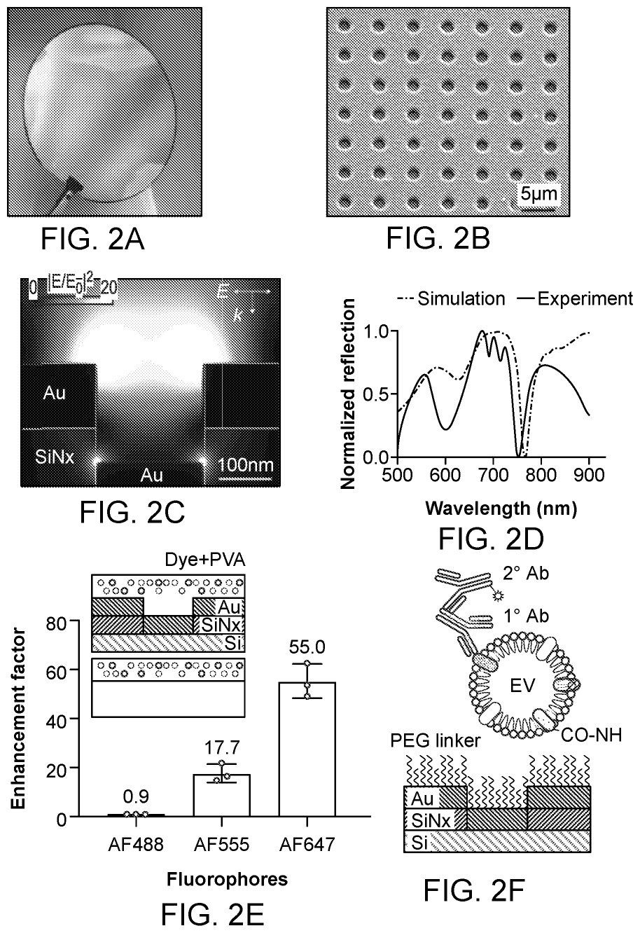

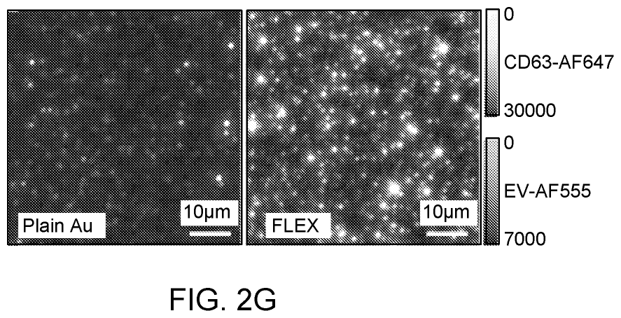

A key concept of the nanowell FLEX technology is the ability to amplify signals from single EVs so that scarce biomarkers can be detected in rare tEVs. The nanowell FLEX sensor was designed with the following considerations: i) nanowells are made in high-throughput, ideally on a wafer-scale through simple fabrication procedures; ii) the fluorescence enhancement range covers the typical EV size ranges (50~200 nm): iii) the resonance wavelengths can be readily tunable and reproducible. As such, periodic gold nanowell structures with 200 nm well diameters and 500 nm periodicity made by interference lithography and metal deposition were designed (FIGS. 2A and 2B). The dimensions were determined by our previous work,^{[17,21]} spectral overlap with commonly used fluorescence dyes, and reproducibility of nanowell fabrication. Interference lithography creates periodic nanowells by exposing a photoresist layer on a Si wafer with two orthogonal grating beams.^{[50]} Subsequent reactive ion etching and gold deposition produce FLEX chips (see FIG. 1C for detailed procedures).

As shown in FIG. 1D, no fluorescence background signal was detected from nanowell chips, while chips with nanoparticles on nanopillars (NPOP) produced random background scattering signals (e.g., possibly from EVs).

FIG. 1E shows a comparison of the coefficients of variations (CVs) of nanoholes and nanowells. Due to simple structure and fabrication processes, nanowells showed about an 8-fold lower CV (4.8% vs. 38.9%) value than nanoholes.

FIG. 2A is a gold nanowell FLEX chip fabricated on a 4-inch Si wafer. The fabricated wafer is diced into 1 cm² chips for EV assays. FIG. 2B is a scanning electron micrograph of the FLEX chip shows the periodic gold nanowell structures. FIG. 2C is a finite-difference time-domain (FDTD) simulation showing the enhanced electromagnetic fields confined near the gold nanowell surface. The enhanced fields are responsible for plasmon-enhanced fluorescence amplification. FIG. 2D shows the reflectance spectra of the gold nanowell arrays with 200 nm diameter and 500 nm periodicity from FDTD simulation (blue dashed line) and experimental measurement (solid red line). FIG. 2E shows the fluorescence signal enhancement of AF488, AF555, and AF647 fluorophores on the FLEX chips compared to a plain glass substrate. We formed a thin polyvinyl alcohol (PVA) layer containing the fluorescence dyes spun-coated on the gold nanowell and glass surfaces, as shown in the schematic in the inset. The data are displayed as mean±standard deviation from triplicate measurements. FIG. 2F is a representative schematic of an EV captured on the gold nanowell surface. The gold surface is functionalized by a polyethylene glycol (PEG) layer to capture EVs. The captured EVs are then labeled by primary (1°) antibodies followed by fluorophore-conjugated secondary antibodies (2°). FIG. 2G is a representative fluorescence image of fluorescently labeled EVs captured on FLEX and glass substrates. For the same sample, EVs captured on the FLEX chip generate stronger fluorescence intensities.

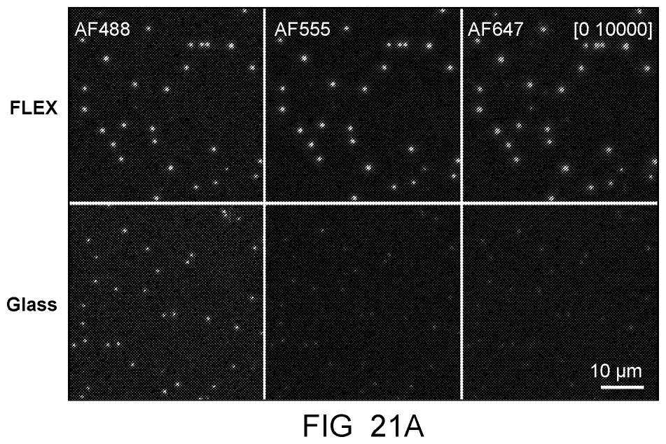

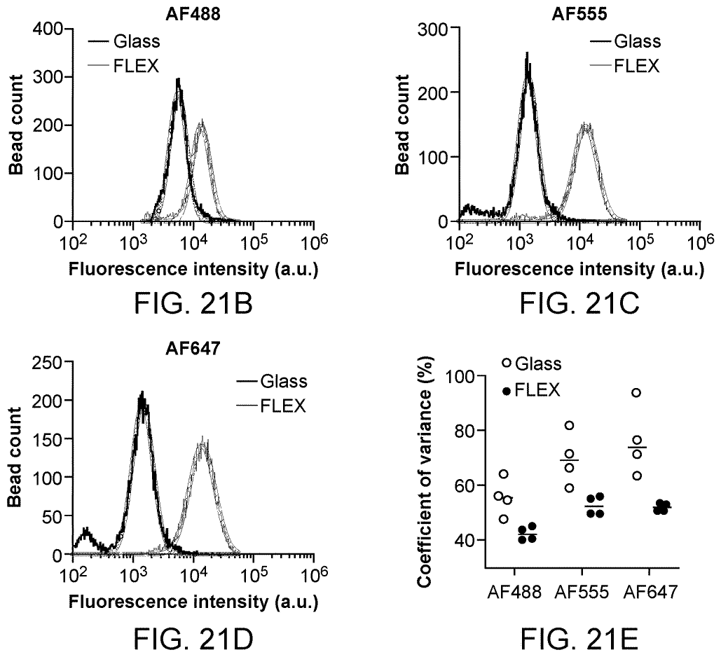

With wafer-scale batch fabrication, one can construct a microarray-type sensing array using a microarray spotter for high-throughput analysis.^{[21]} The periodic nanowell structures support long-range surface plasmon resonances extended to cover small EVs (e.g., exosomes) captured on a gold surface. The finite-difference time-domain (FDTD) simulation for nanowells with 200 nm diameter and 500 nm periodicity shows the strong field enhancement extended in a long range at a resonance wavelength, in addition to the localized enhancement along the nanowell edges (FIG. 2C). The long-range resonance induces homogenous signal enhancements for particles captured within the active area with periodic nanowell arrays, and the signal enhancement does not increase the coefficient of variances for particle intensities (FIGS. 21A-21E). With our simple fabrication process, the optical properties of the fabricated FLEX chip were well-matched with simulation results (FIG. 2D). The resonance wavelengths can also be tuned by adjusting the periodicity of nanowell arrays.

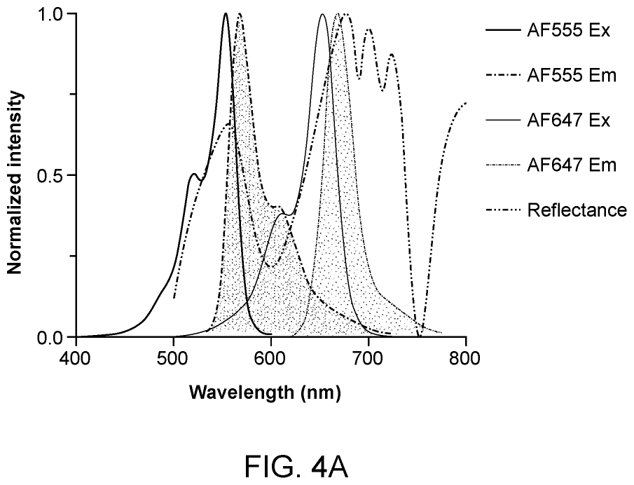

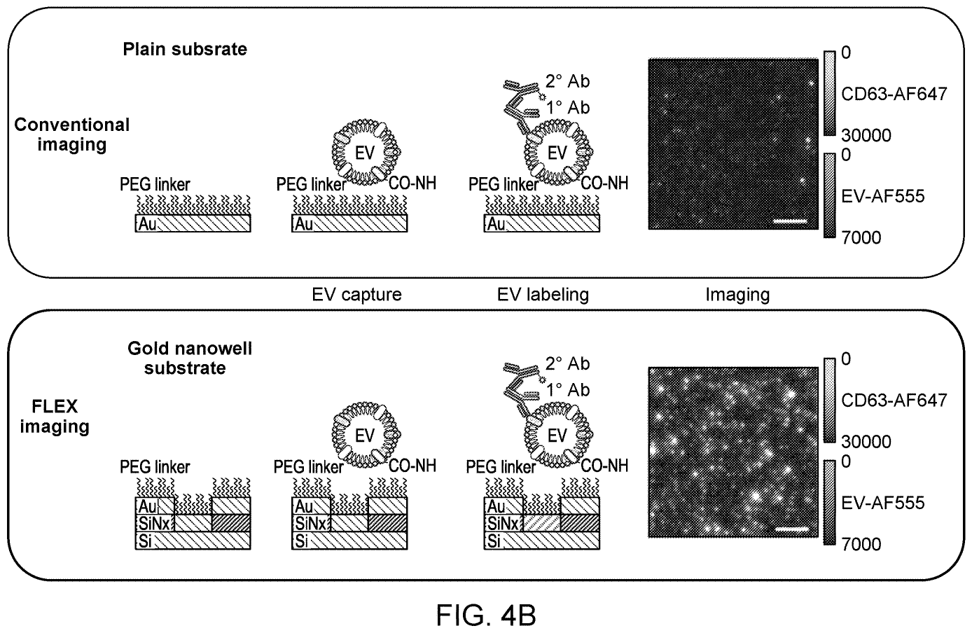

To test the enhancement of fluorescence signals, a thin polyvinyl alcohol (PVA) layer containing fluorescence dyes (AF488, 555, and 647) on the nanowell and glass surfaces were first formed for comparison (FIG. 2E). A 55-fold signal enhancement with AF647 and a 17.7-fold enhancement with AF555 were achieved. The fluorescence signal enhancements are attributed to the spectral overlap between optical resonances of periodic nanowell structures and the excitation and emission spectra of AF555 and AF647 fluorophores (FIG. 4A). In the case of AF488, which has optical spectra off of the plasmonic resonances, it shows comparable intensities between the two substrates. For EV detection, we first functionalized the gold surface with a mixture of 1 kDa carboxylated PEG and 0.2 kDa methylated PEG, which showed the optimal performance for EV capture.^{[17]} After EV capture, target EV proteins were labeled by primary antibodies followed by fluorescently labeled secondary antibodies (FIG. 2F).

Fluorescence images of EVs on the nanowell FLEX chip were imaged using a fluorescence microscope. To demonstrate that the signal enhancement was due to plasmonic resonances induced by periodic nanowell structures, we captured the same EV concentrations and fluorescently labeled them using the same procedures (FIG. 4B). For the FLEX substrate, the underlying gold nanowell structures amplify EVs' fluorescence signals both in AF555 (EV labeling) and AF647 (CD63 labeling) channels, which showed significantly higher intensities and detected EV counts compared to using a plain gold substrate (FIG. 2G). The results showed that using FLEX chips as substrates could significantly improve EV detection sensitivity without requiring extra amplification processes or specialized instruments.

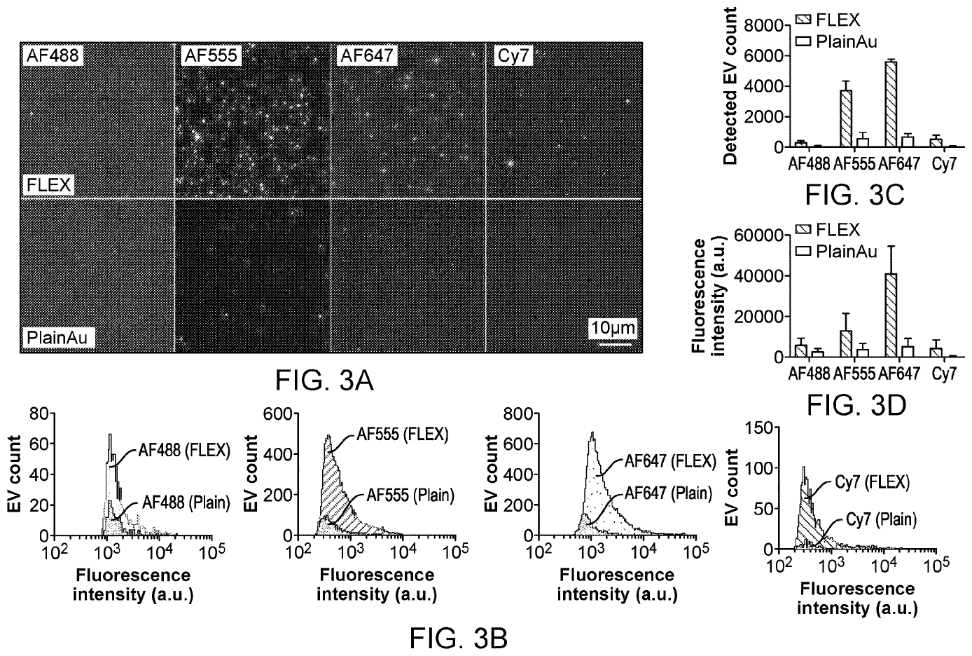

Next, the plasmon enhancements in different fluorescence channels for multiplexed EV analysis were characterized. EVs from a cholangiocarcinoma cell line (SNU308) were fluorescently labeled using tetrafluorophenyl (TFP) ester conjugated with AF488, AF555, AF647, or Cy7 dyes^{[26]} (see Methods and Materials for EV isolation and labeling protocols). The fluorescently labeled EVs were captured on the FLEX and plain gold substrates and compared the fluorescence intensities of individual EVs (FIGS. 3A-3B). Here, plain gold substrate was used as a control for comparison in identical conditions. Although plasmon-enhanced signal amplification is clearly seen with FLEX chips compared to glass substrates, as shown in FIGS. 2E and 21A-21E, by using plain gold substrates, one could use the same material, thickness, surface chemistry, EV samples, and assay procedures for the comparison tests. As mentioned earlier, the Au surface was coated with 1 kDa PEG, which provides enough spacing between captured EVs and gold surfaces to prevent quenching. Nonetheless, if there is any fluorescence quenching on a plain god substrate, the same quenching would happen on the FLEX chip. FIG. 3A shows representative EV fluorescence images of FLEX and plain substrates in the same color scale, and FIG. 3B shows EVs' intensity histograms. The results clearly show the fluorescence signal enhancements of EVs captured on the FLEX chip and significantly improved detection sensitivity in multiple channels. For EVs labeled by AF647, about 5,600 EVs were detected in 250×250 μm² using the FLEX substrate, while less than 700 EVs were detected in the same-sized area using the plain substrate. For EVs labeled by AF555, the detected EV counts were 3.700 with the FLEX and 550 EVs with the plain substrate. As explained earlier, it should be noted that the same material, surface chemistry, and EV amounts were used for both substrates. The results indicate that conventional fluorescence imaging using a plain substrate detected only 10-15% of total EVs without a signal enhancement. This is likely due to weak fluorescence signals with small EVs; these weak signals get amplified on the FLEX chip by the underlying nanowell structures. In the case of EVs labeled by Cy7, a 16-fold higher number of EVs (500 with nanowell vs. 30 with plain) was detected with the FLEX substrate, but the absolute intensities were lower than EVs labeled by AF555 or AF647, which is likely due to the relatively weaker fluorescence signals of the NIR dye. Therefore, we determined to use AF555 for universal EV detection with TFP labeling and AF647 for CCA marker detection.

FIG. 3A shows representative fluorescence images of EVs captured on FLEX and plain Au substrates. EVs were fluorescently labeled by AF488, AF555, AF647, and Cy7 fluorophores, respectively, and the same amount of EVs were captured on the gold surfaces using the same PEG linkers. FIG. 3B are histograms of detected EV counts based on their fluorescence intensities. Colored bars represent EVs captured on FLEX chips, and gray bars represent EVs captured on plain Au substrates. FIGS. 3C-3D shows numbers (C) and fluorescence intensities (D) of detected EVs with FLEX and plain Au substrates. EVs were fluorescently labeled by AF488, AF555, AF647, and Cy7 fluorophores, respectively. The data are displayed as mean±standard deviation from triplicate measurements.

FIG. 4A shows that the reflectance spectrum of the periodic nanowell array (200 nm in diameter and 500 nm periodicity) overlapped with the excitation and emission spectra of AF555 and AF647 fluorophores.

FIG. 4B is a comparison between conventional EV fluorescence and FLEX imaging. As shown in FIG. 4B, the overall procedures (substrate preparation, EV capture, labeling) are the same, but the only difference is using a gold nanowell substrate instead of plain gold or glass substrates. The gold nanowell substrate, however, significantly amplifies EVs' fluorescence signals, detecting a larger number of EVs than plain substrates.

FIG. 5A shows representative two-channel fluorescence images of SNU308 EVs. All captured EVs are labeled by AF555 (green), and EpCAM was detected by immunolabeling with anti-EpCAM antibody followed by AF647 secondary antibodies. FIG. 5B shows fluorescence intensity line profiles in AF555 and AF647 channels along the white dashed line shown in FIG. 5A. AF555 represents EV signals, and AF647 represents EpCAM signals. FIGS. 5C-5F show 2D scattering plots of EVs from a SNU308 cell line. The x and y axes represent AF555 (universal EV staining) and AF647 (cancer biomarker) intensities, respectively. When AF555 and AF647 intensities are colocalized, EVs are counted. The graphs on the right side represented the colocalization ratios of EpCAM, MUC1, EGFR, and IgG (isotype control). The marker threshold value dashed lines along the y-axis were defined by mean±3×standard deviation of IgG signals.

The plasmon-enhanced detection enables sensitive detection of single EVs and their markers, otherwise undetected by their weak detection signals. This is especially useful to detect EVs and their markers in low abundance. We could detect less than 15% of the EVs present in samples without the signal enhancement.

The plasmon enhancements also occurred at multiple wavelengths, enhancing signals above 500 nm. This allows us to do multichannel imaging and expand our channel to NIR wavelengths that are barely used in EV imaging due to weak fluorescence signals.

The multichannel imaging and colocalization analysis ensure the detected particles are marker-positive, tumor-derived EVs, while the specificity of single-channel imaging and analysis could be affected by nonspecific interactions or background signals. This also enables us to accurate enumerate our target marker-positive EVs and their marker intensities in single vesicles.

### Example 2: Single EV Analysis of CCA-Derived EVs

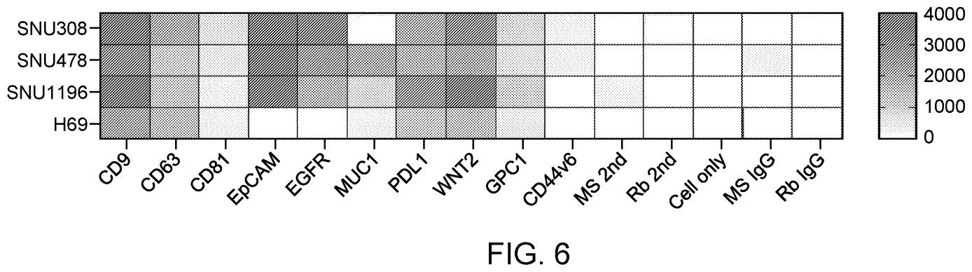

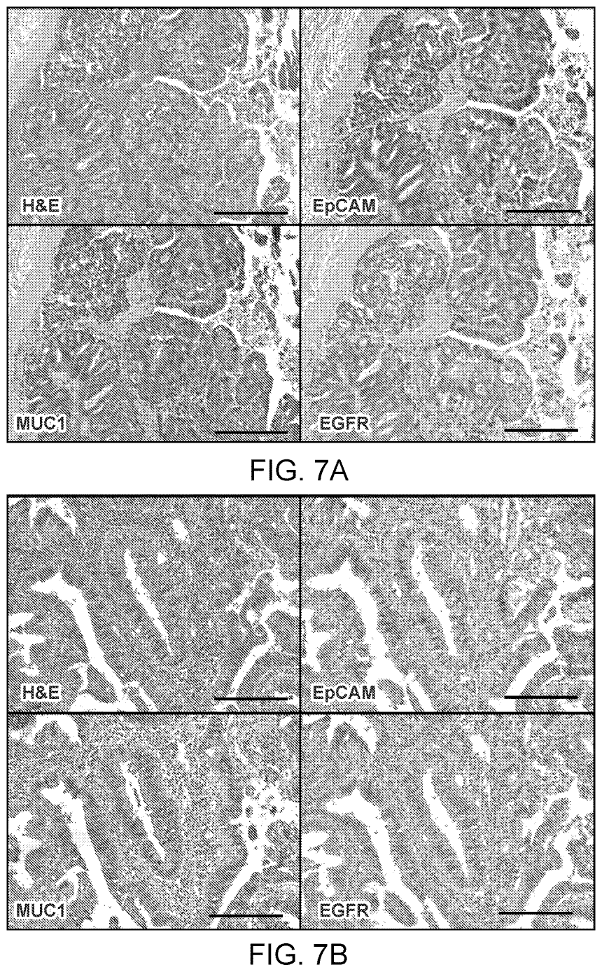

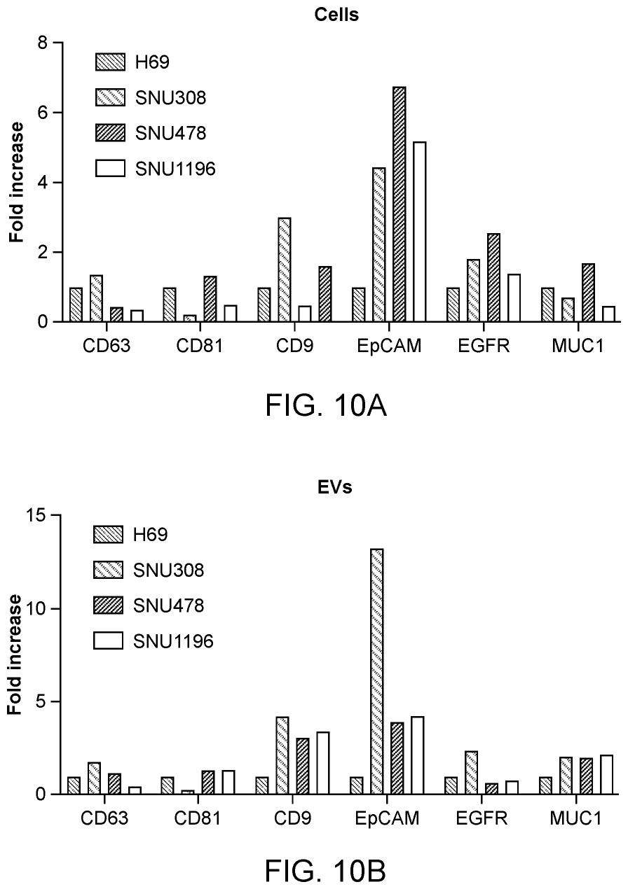

We reviewed the literature on CCA markers^{[51-63]} and compared candidate CCA markers with those present in benign cells. This analysis led to the following candidate markers: EpCAM, EGFR, MUC1, PD-L1, WNT2, GPC1, and CD44v6. Our cellular analysis on these candidate markers and EV putative markers (CD63, CD9, and CD81) was first performed using CCA cell lines (SNU308, SNU478, and SNU1196) and normal human cholangiocyte cell line (H69). The flow cytometry analysis showed that EpCAM, EGFR, and MUC1 are over-expressed in CCA cell lines, and importantly, the levels of these three were negligible in the control H69 cell line (FIG. 6), which was not true for other candidates. The over-expressions of EpCAM, EGFR, and MUC1 in CCAs were also validated by conventional immunohistochemistry (IHC) staining of tissue samples from CCA patients (FIGS. 7A-7B) and proteomic analyses (FIGS. 10A-10B).

The three markers across EVs derived from CCA cell lines were then measured and we investigated their heterogeneity from single EV analyses with nanowell FLEX chips. After EV isolation from cell culture supernatants, isolated EVs were labeled by TFP-AF555. Then, ~5×10⁵ EVs diluted in PBS were captured on the FLEX chip surface following NHS/EDC activation. The captured EVs were then immunolabeled with primary antibodies for target cancer and EV markers (EpCAM, MUC1, and EGFR), followed by labeling with AF647-conjugated secondary antibodies. FIGS. 5A and 5B show a representative fluorescence image and corresponding intensity profiles of SNU308-derived EVs in the TFP (AF555) and marker (AF647) channels.

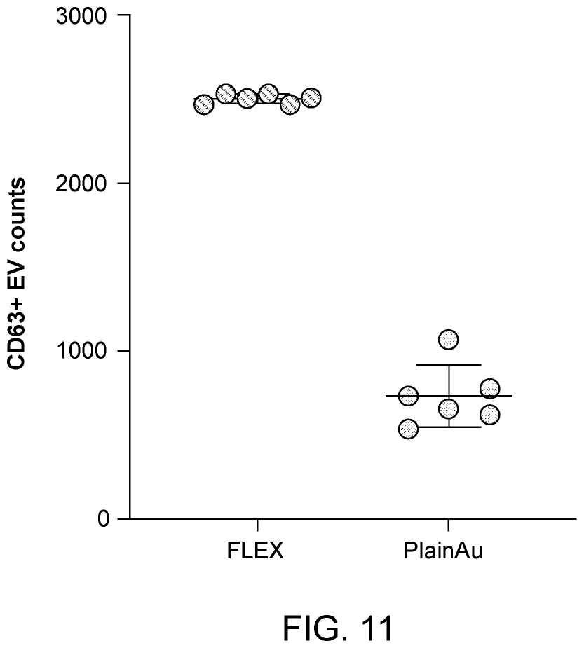

In the analysis, EV positions in the AF555 channel were first located by detecting spots with a signal-to-noise ratio (SNR) greater than 3. At each detected EV position, the cancer marker intensity in the AF647 channel was measured. In this way, false-positive marker signals due to the nonspecific binding of antibodies to the sensing surface was minimized. Furthermore, the marker positivity threshold was set using an IgG control where the threshold value was defined by the mean±3×standard deviation of IgG signals. For each marker, 4 images in which the field of view of each image was about 250×250 μm² covered by 1 μl EV solution was obtained. On average, about 7437±636 (n=27) EVs per field of view were detected and analyzed. For the same amount of EVs applied, one can see significantly higher numbers of TFP- and CD63-positive EVs using the FLEX substrate than a plain substrate (FIG. 11).

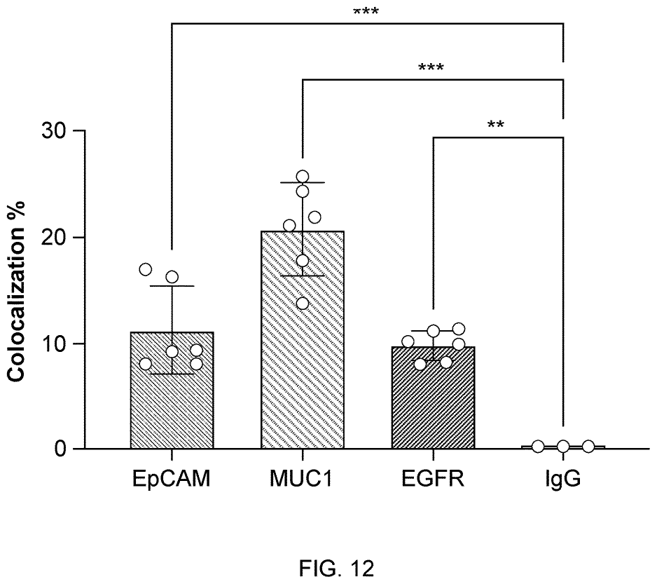

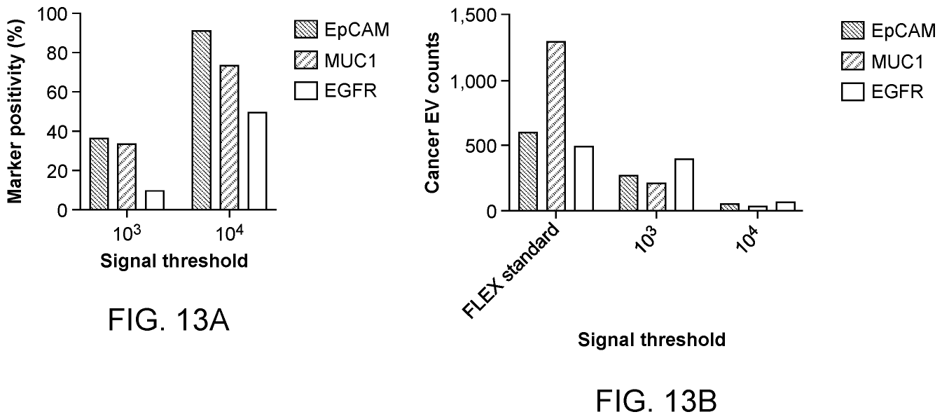

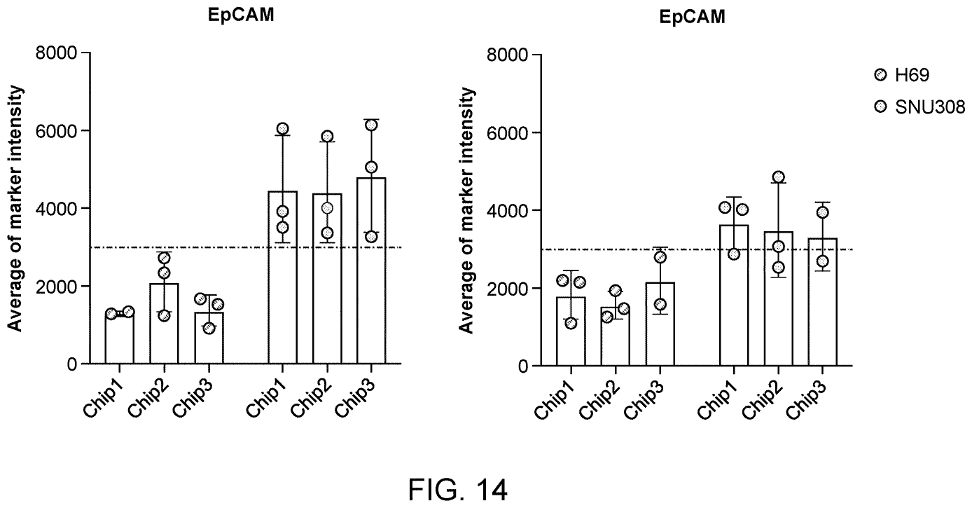

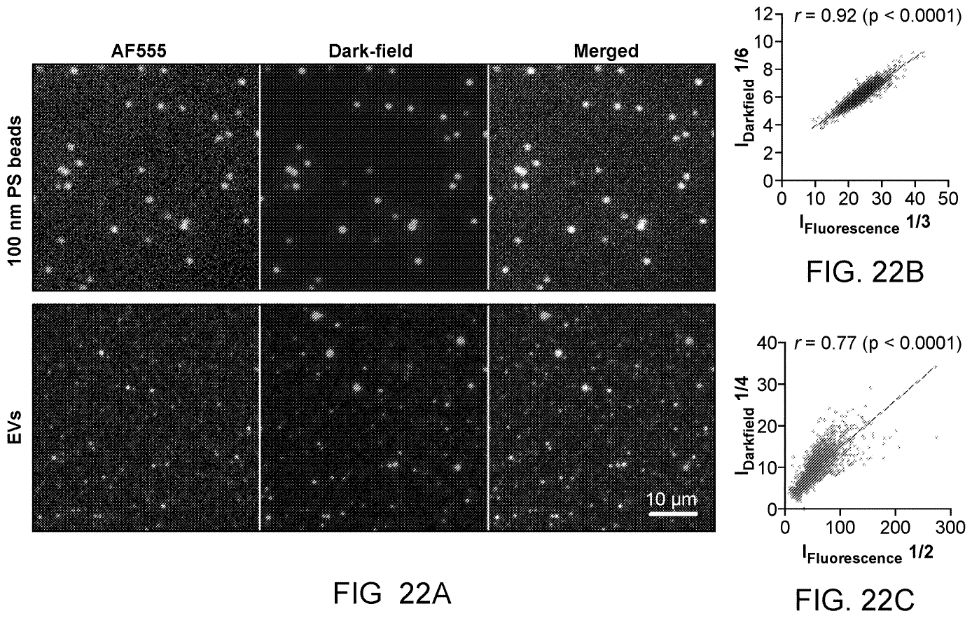

Interestingly, for each of the three tumor markers (EpCAM, EGFR, and MUC1), only 10-20% of total EVs contain all tumor markers with a wide range of expression levels and a more than one-order difference (FIGS. 5C-5F). In contrast, all three markers are present in the CCA cell line-derived EVs with significantly higher ratios than an IgG control (P<0.001, one-way ANOVA with Dunnett's multiple-comparison test, FIG. 12). The false-positive rate in the IgG control channel was measured at less than 0.5%. The marker positivity rates substantially increased for EV subpopulations with strong TFP signals (FIGS. 13A-13B). For example, the rate of MUC1-positive EVs increased to 33% for EVs with TFP-AF555 signals greater than 10³ and 74% for EVs with TFP-AF555 signals greater than 10⁴. However, with no thresholds, 33- and 6-fold higher numbers of MUC1-positive tEVs were detected compared to the 10⁴ and 10³ threshold cases, respectively. The TFP signal intensities, in general, correlate with dark-field scattering light intensities of EVs (FIGS. 22A-22C). This indicates that most EVs were in small sizes (weak fluorescence intensity), which could be missed without signal amplification. Without the enhancement, it could also provide biased analysis by analyzing larger (or aggregated) EVs with strong signals. These differences can be critical for early cancer detection, where tEV counts are relatively low. The assay also shows good reproducibility with a chip-to-chip coefficient variation below 5% (FIG. 14).

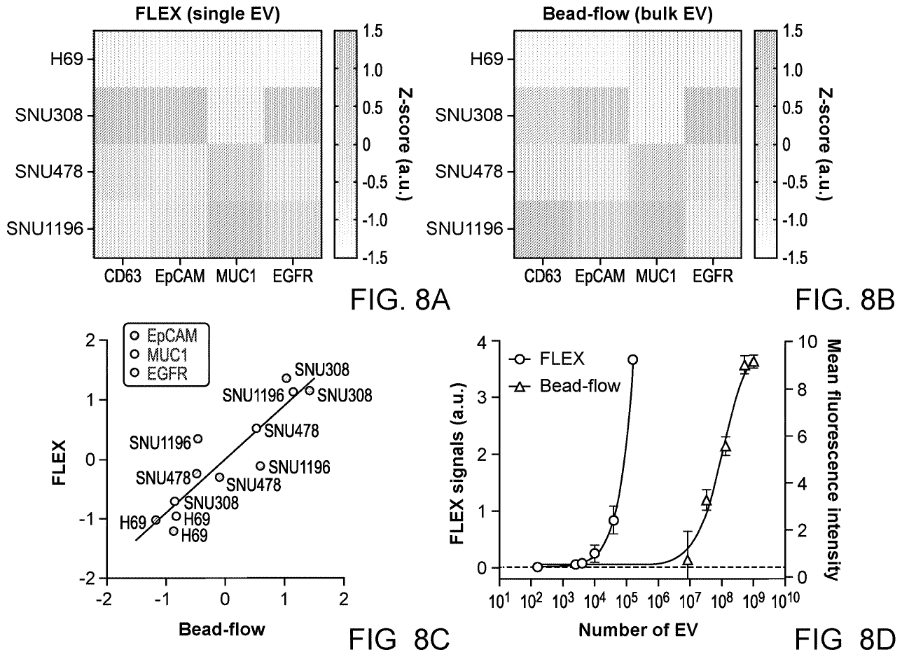

The analysis was extended to other cell line-derived EVs and the results were compared with bulk EV analysis using bead-based flow cytometry (FIGS. 8A and B). Bead-based flow cytometry was chosen because the method showed higher sensitivity than other bulk EV methods (ELISA and Western blot)^{[64]} and can be run with a similar configuration to the FLEX assay; EVs were immobilized on the bead surface, followed by labeling using the same sets of primary and fluorescent secondary antibodies. The heatmaps showed the z-scores of normalized marker levels compared to isotype controls. Linear regression analysis showed a good linear correlation (Pearson r=0.91) between the single EV analysis and the bulk EV approach using flow cytometry (FIG. 8C). The bead-based bulk EV analysis with flow cytometry achieved a limit of detection of 7.5×10⁷ EVs, whereas the FLEX technology achieved a limit of detection of 2×10³ EVs (FIG. 8D).

Here, the aliquots of samples in the same volume (30 l) were used for direct comparison. The limit of detection indicates the amount of EVs required for reliable detection to determine marker positivity. This is mainly limited by the diffusion of EVs to the sensor surface for capture. Thus, by reducing the diffusion time, a lower required amount was expected. Applying a microfluidic channel with herringbone patterns^{[31]} or field-induced mixing^{[65][69]} could potentially reduce the diffusion time, leading to a lower detection limit. Combined with plasmon enhancement, the 4-orders of magnitude increase in sensitivity could enable the detection of rare tumor-derived EVs in clinical samples and their protein markers.

This example shows that MUC1. EGFR, and EpCAM are highly expressed in EVs from CCA cells or tissues. It also shows that not all EVs from the same source contains the same levels of markers, thus quantification of marker-positive EVs are important. It also shows the identified markers are found both EVs and their parental cells, supporting the use of EVs as surrogate markers.

### Example 3: EV Analysis of Clinical Samples for CCA Diagnostics

Next, we applied the nanowell FLEX assay in a pilot study to detect CCA-derived EVs using clinical samples. Bile samples (4-14 mL, mean=6.8 mL) were collected from CCA (n=17) and non-cancer patients (n=8) by ERCP. Different EV isolation methods for bile samples were first tested, including the gold standard ultracentrifugation, size-exclusion chromatography (SEC), and their combination. Ultracentrifugation (UC) is a preferred method for a large volume of samples with a high isolation capacity. On the other hand. SEC isolates EVs based on their unique size range, larger than soluble proteins and smaller than cell debris.

FIGS. 7A-7B show Hematoxylin-eosin (H&E) and EpCAM, MUC1, and EGFR immunohistochemical staining of two patients (A, B) show high expression of the three markers in the tissue of cholangiocarcinoma patients. Scale bars=200 μm.

FIGS. 8A-8B show that CCA (EpCAM, MUC1, and EGFR) and EV (CD63) markers were profiled for EVs from three different CCA cell lines and one normal cell line by single EV analysis with FLEX (A) and bulk EV analysis with bead-flow assays (B). FIG. 8C shows the correlation of z-scores for each marker between FLEX and bead-flow measurements. The Pearson correlation coefficient r=0.91 (P<0.0001).

FIG. 8D shows the comparison of the detection sensitivity of FLEX and bead-flow assays. Aliquots of samples in the same volume (30 l) were used for direct comparison. The detection limits were determined by titrating known EV quantities and measuring their CD63 signals. The data are displayed as mean±standard deviation from duplicate measurements.

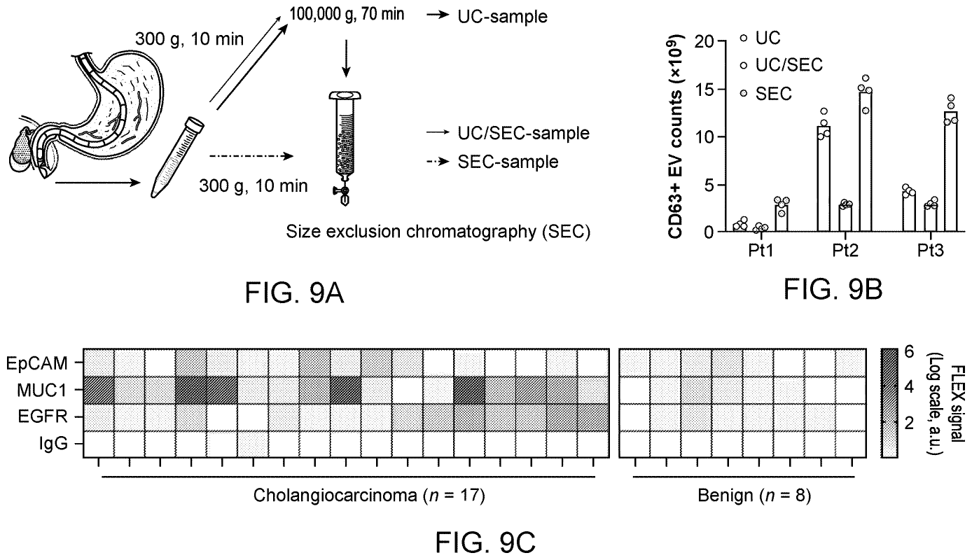

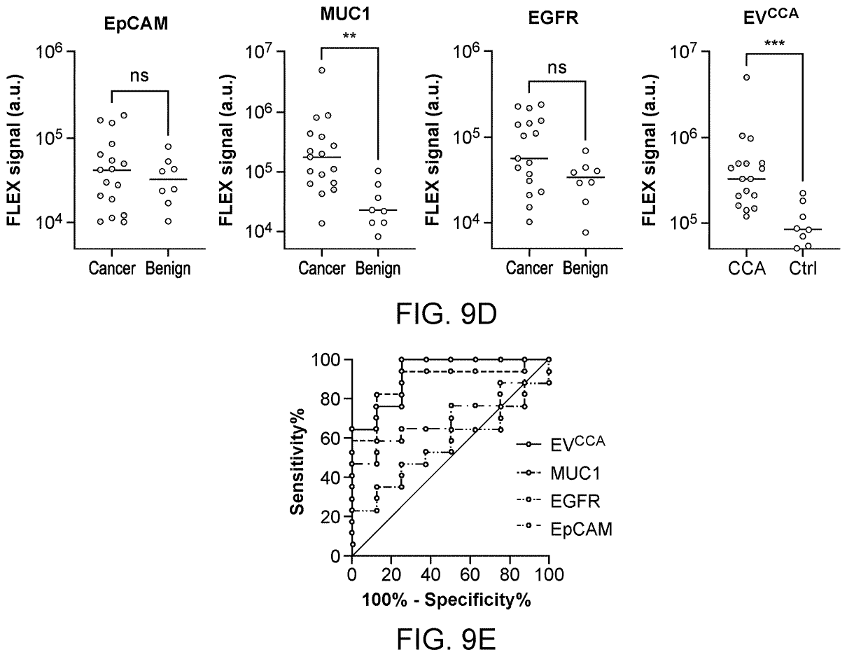

FIG. 9A shows the EV isolation from human bile samples. Ultracentrifugation (UC), size exclusion chromatography (SEC), and their combination were tested. FIG. 9B shows the comparison of CD63-positive EV counts. EVs were isolated from three patient bile samples using methods depicted in A. SEC method showed the highest CD63-positive EV counts for all three samples tested. The data are displayed as mean±standard deviation from quadruple measurements. FIG. 9C shows the analysis of bile-derived EVs from 17 CCA and 8 benign patients for selected CCA markers (EpCAM, MU1, and EGFR). IgG isotype was used as a negative control. The FLEX signals from each marker are represented in a heatmap on a log scale. FIG. 9D shows the FLEX signals on each tEV marker (EpCAM, MUC1, EGFR) and their combination (EV^{CCA}) as measured in bile-derived EVs from 17 CCA and 8 benign patients. Mann-Whitney unpaired t-test was done (**P<0.01 and ***P<0.001). FIG. 9E shows the receiver operating characteristic curves of each tEV marker and intensity and EV^{CCA}. The EV^{CCA} signature showed the highest area-under-the-curves (AUC) value of 0.93.

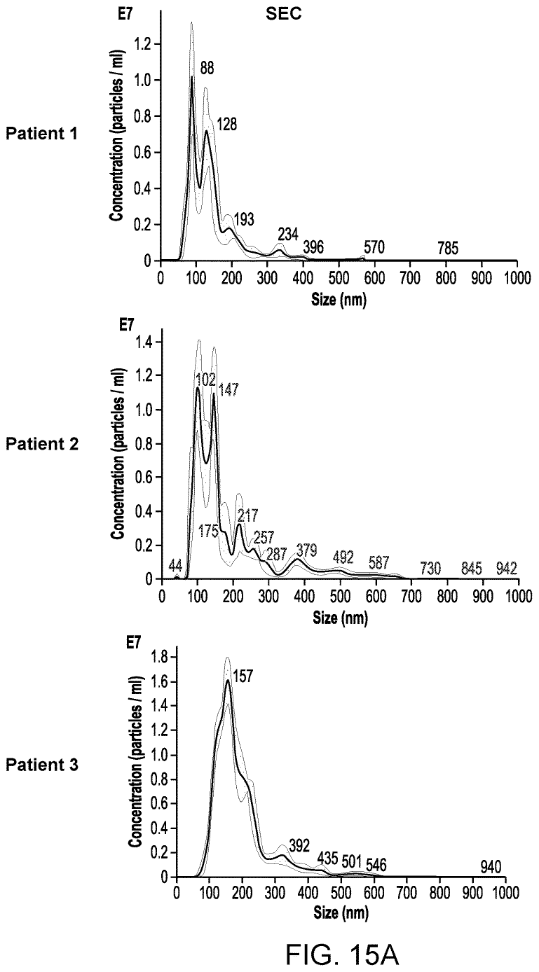

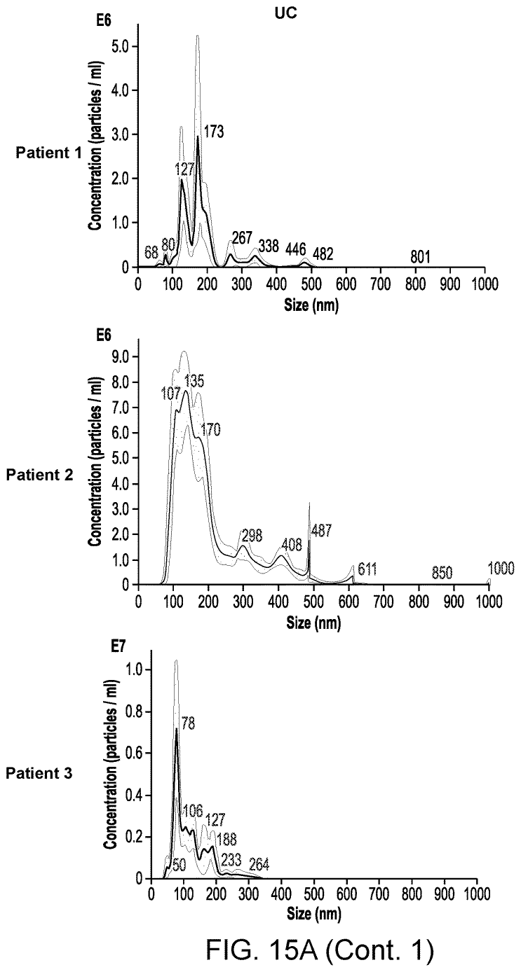

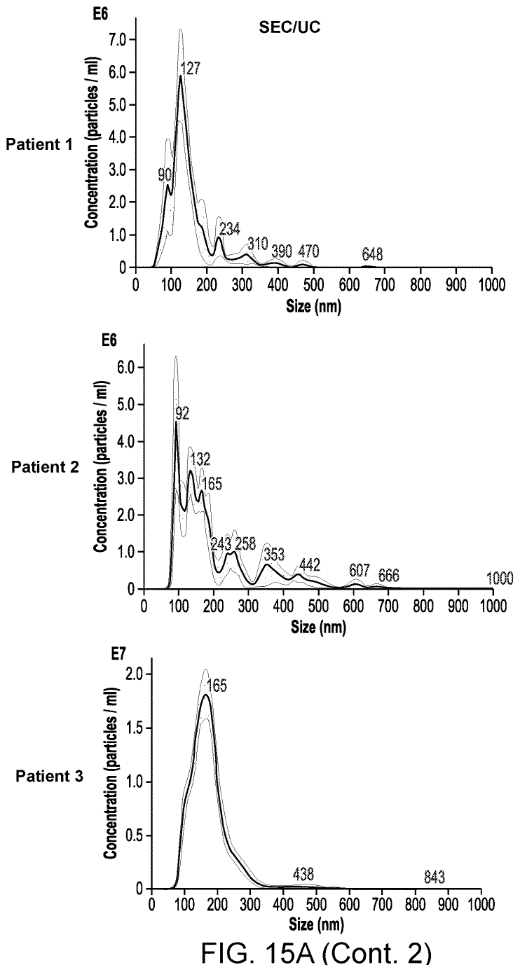

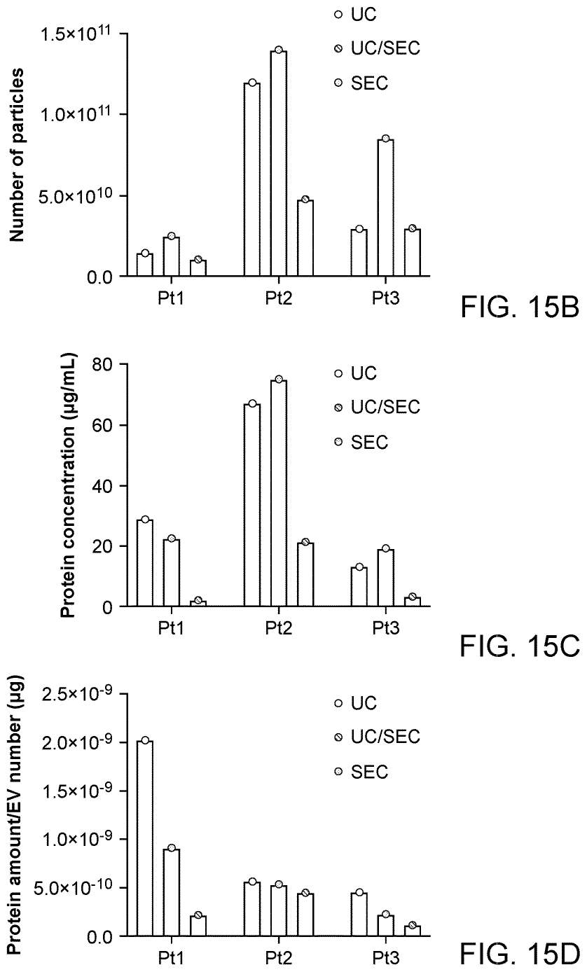

After removing floating dead cells and debris by centrifugation at 300 g, EVs from 1 mL of bile samples (n=3) were isolated using the three methods (FIG. 9A) and evaluated CD63-positive EV counts, total EV counts, and protein levels (FIGS. 9B and 15A-15D). SEC showed better EV isolation efficiency in total and CD63-positive EV counts than UC and UC/SEC combination.

Interestingly, the protein amounts per EV were higher in UC than in SEC. This result may represent the major weakness of UC isolation for protein contamination in isolated EVs. The UC/SEC combination showed 5-fold lower CD63-positive EV counts than using SEC only. This can imply that the combination method led to a significant loss of EVs through the two-step process. Based on these results, we decided to use SEC for EV isolation from bile samples.

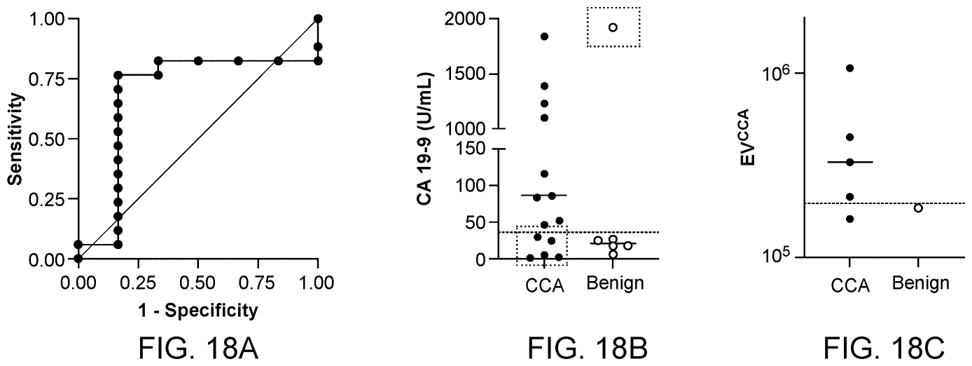

For FLEX signals, the total fluorescence intensities of marker-positive EVs was calculated to take into account both the tEV count and their marker levels. For marker-positive EVs, MUC1 showed significantly elevated signals from CCA patients compared to benign patients (P=0.001 for MUC1, Mann-Whitney unpaired t-test. FIG. 9D), but the area under the curve (AUC) remained below 0.9. While EpCAM and EGFR do not show significant increases in EVs from CCA patients as single markers, their combinations with MUC1 (we termed “EV^{CCA}”) further increased the difference between CCA and benign groups (P=0.0003, Mann-Whitney unpaired t-test) and increased the AUC to 0.93 (FIG. 9E). This is because adding EGFR and EpCAM allowed us to detect cases with low or no MUC1 expressions. The AUC of CA19-9 was only 0.69, and the diagnostic accuracy with a clinical threshold value of 37 U/mL was 74% (17/23), similar to previously reported ranges (FIGS. 18A-18C)^{[5,6]}. Among the six patients with abnormal CA 19-9 values, five were correctly discriminated using EV^{CCA}.

As shown in FIGS. 10A-10B, the LC/MS analysis shows elevated levels of EpCAM, EGFR, and MUC1 in cholangiocarcinoma cell lines (SNU308, SNU478, SNU1196, A) and their EVs (B). The data were normalized and shown as fold change over the marker level of the normal bile duct cell line (H69) and their EVs, respectively.

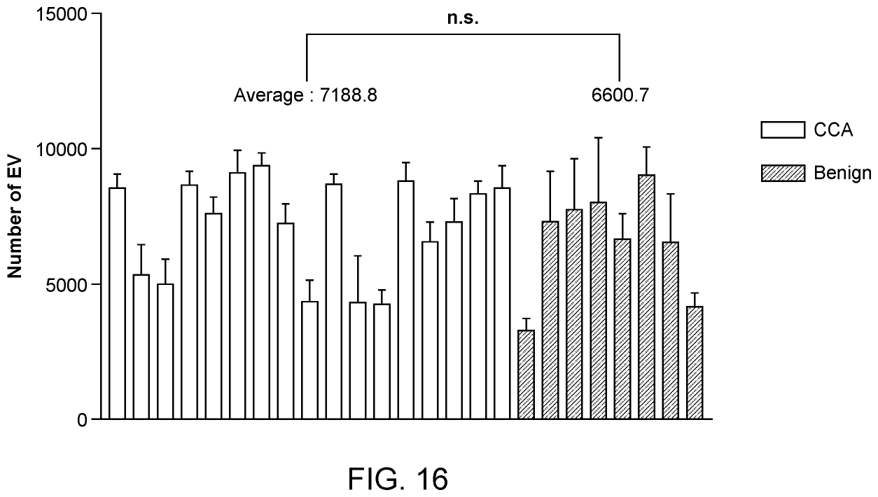

The nanowell FLEX analysis was expanded to 25 patients (n=17 for BTC patients and n=8 for benign patients) using EpCAM, MUC1, and EGFR (FIG. 9C). The clinical details of these patients are described in Table 1, below. While a previous study showed elevated EV counts in CCA patients^{[15]}, in this cohort, a significant difference in total EV counts between the two groups (FIG. 16) was not seen. However, a different differential pattern from marker-positive tEVs could be seen.

TABLE 1
Clinical Information of Patients

| | | | Non-maligant | |
|---|---|---|---|---|
| | Cholangiocarcinoma | | obstruction | |
| Characteristic | (n = 17) | | (n = 8) | |
| Primary location of maligancy (%) | | | | |
| Perihilar bile duct | 5 | (29.4) | — | |
| Distal bile duct | 12 | (70.6) | — | |
| Cause of biliary obstruction (%) | | | | |
| Biliary stones | — | | 7 | (87.5) |
| Benign biliary stricture | — | | 1 | (12.5) |
| Age, yr | | | | |
| Median (range) | 71 | (37-80) | 67.5 | (50-80) |
| Sex (%) | | | | |
| Male | 10 | (58.8) | 4 | (50) |
| Female | 7 | (41.2) | 4 | (50) |
| Bile sample volume (mL) | | | | |
| Mean (min, max) | 6.9 | (4, 14) | 6.4 | (5, 8.5) |
| CA19-9, U/mL | | | | |
| Mean (SD) | 1606.5 | (4776.7) | 336.6 | (777.2) |
| Total bilirubin, mg/dL | | | | |
| Mean (SD) | 7.5 | (5.7) | 3.4 | (3.1) |
| CRP, mg/L | | | | |
| Mean (SD) | 30.1 | (26.8) | 40.0 | (42.0) |

| Stage of tumor (%) | | | |
|---|---|---|---|
| I | 3 | (17.6) | — |
| II | 8 | (47.1) | — |
| III | 0 | (0.0) | — |
| IV | 6 | (35.3) | — |
| Pathological confirmation | | | |
| methods (%) | | | |
| 1st ERCP sampling | 12 | (70.6) | — |
| 2nd ERCP sampling | 1 | (5.9) | — |
| Surgery | 2 | (11.8) | — |
| Other method* | 2 | (11.8) | — |

Abbreviation: SD, standard deviation; ERCP, endoscopic retrogrde cholangiopancreatography.
*Liver biopsy and ascites cytology were used.

As shown in FIG. 12, EVs from the SNU308 cell line show significantly higher positive rates for EpCAM, MUC1, and EGFR than IgG isotype control. P values were calculated by Mann-Whitney unpaired t-test (**P<0.01 and ***P<0.001).

FIG. 13A shows the marker positivity rates increase with a higher signal threshold value (i.e., only considering EVs with high AF555 signals). FIG. 13B shows the detected tEV counts with the standard threshold in the FLEX assay (defined by mean±3 times standard deviation of IgG signal) and higher thresholds (103 and 104). Fluorescence and dark-field scattering images of 100 nm polystyrene (PS) beads and EVs labeled by TFP-AF555. The Pearson correlation coefficients are shown on the plot with p-values. The red dot lines show linearly fitted curves.

As shown in FIG. 14, three duplication measurements were performed on three different chips for EpCAM detection on EVs from SNU308 and H69 cell lines. Two independent measurements were performed for a reproducibility test. The coefficient of variation (CV) from three different chips was 4.8% for positive controls, while negative controls were below threshold values. The specificity for EpCAM measurements was reproduced in the two independent tests. Each experiment was replicated 3 (n=3).

FIG. 15A shows the nanoparticle tracking analysis of bile-derived EVs from three patients using size-exclusion chromatography (SEC), ultracentrifugation (UC), and their combination (SEC/UC). FIGS. 15B-15D show the numbers of total particles (B), protein concentrations (C), and protein amount (μg) per EV for bile-derived EV samples were isolated by the three methods.

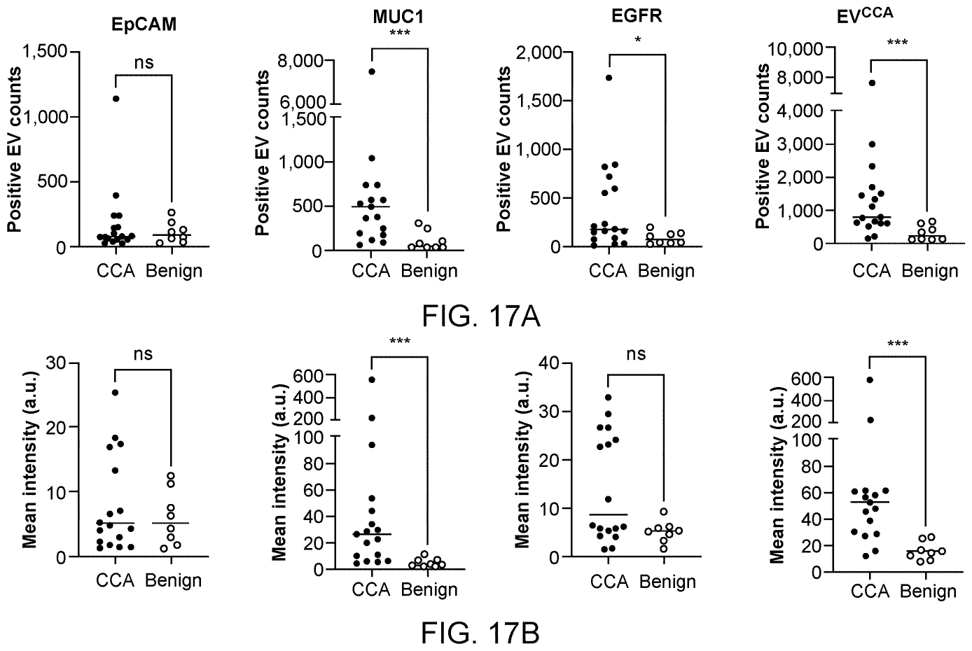

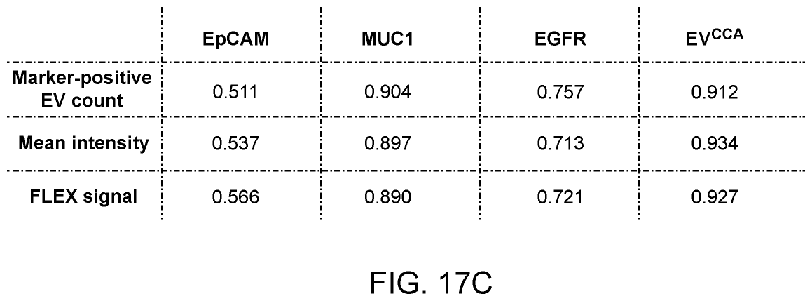

FIG. 17A shows the marker-positive EV counts. FIG. 17B shows the mean intensities of marker-positive EVs. FIG. 17C shows the area under the curve (AUC) values for marker-positive EV count, mean intensity, and FLEX signal (i.e., total intensities of marker-positive EVs). P values were calculated by Mann-Whitney unpaired t-test (***P<0.001).

Here, tEVs were detected based on marker positivity and measured their counts and individual EVs' intensities (FIGS. 17A-17C). FIG. 18A shows the receiver operating characteristic (ROC) curve for CA19-9. FIG. 18B shows the CA19-9 levels between CCA and benign groups. FIG. 18C shows the FLEX analysis using EVCCA signature for six patients misdiagnosed with CA19-9 highlighted as blue dashed boxes in (B). 5 out of 6 patients were correctly diagnosed with the FLEX assay.

FIG. 19A shows transmission electron micrograph of isolated EVs. FIG. 19B shows expressions of EV marker (CD63, Flotilin-1, CD9) and EV tumor markers (EpCAM, MUC1, EGFR) measured with Western blot with EV lysis. FIG. 19C shows the EV concentrations and size distributions measured with nanoparticle tracking analysis (NTA). FIG. 19D shows the average EV concentrations were summarized for cell line-derived EVs (n=4). The presence of background particles in the PBS buffer solution was also investigated. From 5 repeated measurements, three measurements showed no particle presence, and two measurements showed only 0.2 particles per frame. The calculated average concentration was below the limit of detection of particles of NTA (1.7×107 particles/mL, NanoSight LM-10, Steppert et al., J. Chromatogr A. 2017; 1487:89-99). We, therefore, concluded EVs are non-detectable (N.D.) in the PBS solution.

FIG. 21A shows fluorescence images of beads placed on glass and FLEX substrates in AF488, AF555, and AF647 channels. FIGS. 21B-21D. Histogram analysis of fluorescence intensities in (B) AF488, (C) AF555, and (D) AF647. E. Coefficients of variance of bead intensities on glass and FLEX substrates.

In testing and optimizing the TFP labeling, we established a correlation between TFP signals and dark-field light scatterings of EVs. The dark-field scattering was measured using the CytoViva darkfield illumination system and a 40× NA 0.9 objective. As shown in FIGS. 22A-22C, TFP-AF555 signals are aligned well with dark-field light scattering for both polystyrene (PS) beads and EVs. We used different metrics for their correlation because of the structural difference between PS beads and EVs. PS beads have uniform materials and dye distribution in the sphere, while EVs are considered hollow shell structures (Vogel et al., J. Extracell Vesicles, 2021:10:e12052). Therefore. EVs' scattering intensities follow Rayleigh-Gans approximation (I∝d4), while beads' scattering intensity scales with d6. For fluorescence intensity, TFP labeling applies to the surfaces for EVs (d2), while fluorescence beads contain the dyes uniformly in the spheres (d3). Thus, we made scattering plots of Idark1/6 vs. IFluorescence1/3 for PS beads and Idark1/4 vs. IFluorescence1/2 for EVs. The Pearson correlation coefficients (r=0.92 for beads and r=0.77 for EVs) show good correlations between fluorescence and dark-field intensities that are proportional to the size of particles.

### CONCLUSIONS

EVs are attractive circulating biomarkers with their abundance, stability, and molecular cargos from originating cells. Namely, molecular analysis of tumor-derived EVs in biofluids can offer a liquid biopsy-based molecular diagnosis of cancer. The FLEX technology was developed to better facilitate tumor-derived EV detection and molecular analysis that can be integrated into the clinical workflow. The FLEX technology significantly improves the EV detection sensitivity using plasmonic enhancements of fluorescence signals with periodic gold nanowell structures. For the same EV aliquots, the FLEX chip detected an 8-fold higher number of EVs than conventional plain substrates. This indicates that almost 90% of EVs could be missed by traditional immunofluorescence labeling and imaging. However, the FLEX approach achieved high sensitivity to the single EV level without requiring specialized instruments or other signal amplification processes. The high sensitivity is particularly crucial to detect rare EV targets (e.g., scarce tEVs from small sizes of tumors at early stages, phosphorylated or mutated proteins in EVs^{[26]}). The cancer-specific mutation marker detection in tEVs could help improve the diagnosis specificity. Furthermore, as shown in FIGS. 5A-5F, the enhancement could occur in multiple channels (AF555, AF647, Cy7), boosting the sensitivity compared to conventional fluorescence imaging. It is because the periodic nanowell structure supports multiple resonances in different wavelengths. While we did not fully take advantage yet in this work, the plasmonic enhancement would be helpful to extend the multiplexing capability in NIR channels that are barely used in conventional EV fluorescence imaging due to weak signals. This could lead to a higher degree of multiplexing.

During the development of FLEX technology, simple, robust, and wafer-scale fabrication of FLEX chips, which is a key component to achieving higher detection sensitivity with good reproducibility, was the focus. The low-cost, high-throughput chip fabrication often becomes a critical bottleneck when translating highly sensitive plasmonic sensing technologies into clinical applications. Periodic nanowell structures in 4-inch Si wafers using interference lithography and metal evaporation could be produced. The current process costs about $750 per wafer or $12.5 per 10 mm by 10 mm chip. The cost is expected to significantly scale downward with bulk production. Alternatively, nanoimprint or deep ultraviolet (DUV) lithography could be used to pattern periodic nanowells on a wafer scale. Using DUV lithography could provide us more flexibility to further optimize nanowell structures, sizes, and periodicity to maximize the field enhancements and enable a higher degree of multiplexing. The high-throughput, low-cost chip fabrication will open up opportunities to adapt the signal enhancement in other advanced microscopic techniques.^{[45,46]}

CCA is a highly heterogeneous malignancy with multiple subtypes based on its anatomical location along the biliary tree. Therefore, a combination of markers was identified rather than relying on single markers for CCA detection. Through bioinformatic survey and cross-references with EV and protein marker databases, the candidates were narrowed to 10 markers, including 7 tumor-associated (EpCAM. EGFR, MUC1, PD-L1. WNT2, GPC1, and CD44v6) and 3 EV putative markers (CD63, CD81, CD9). Through proteomics and flow cytometry analysis on tissues, cells, and EVs, the diagnostic marker EV^{CCA} signature (MUC1, EpCAM, EGFR) was constituted. Applying the EV^{CCA} signature to bile samples from 25 patients, the nanowell FLEX EV analysis showed high classification accuracy (AUC=93%), which could be attributed to the release of tEV in bile samples and FLEX's high sensitivity in detecting tEVs from a vast background of non-tumor EVs. It should be noted that the sensitivity of an ERCP sampling with conventional clinical pathology remained at 70% (Table 1); the other 30% required additional procedures for confirmation. The EV analysis of bile samples obtained during ERCP procedures could be an excellent complementary test to reduce non-diagnostic cases. We identified tumor markers (MUC1, EGFR, and EPCAM) in CCA and applied this marker combination to detect tEVs in clinical bile samples. The FLEX assay detected CCA with an area under the curve of 0.93, significantly better than current clinical markers. The sensitive and accurate nanoplasmonic EV sensing technology could aid in early CCA diagnosis.

The current methodology can be applied to blood samples. Compared to other cancer types, there are few studies on CCA detection through EV analysis. The current bile EV analysis was important to identify the presence of tumor-derived EVs in biofluids, analyze their protein signatures, and the correlation with originating tumors. Blood samples have been used for other cancer types, including pancreatic cancer^{[17,21,26,64]}. Notably, using blood samples could allow the serially assessment of patients and enable treatment monitoring. The sensitive single EV analysis using FLEX could facilitate the detection of scarce tumor-derived EVs and quantify their temporal changes in cancer development and therapy responses.

### REFERENCES

[1] R. L. Siegel, K. D. Miller, A. Jemal. CA: Cancer J. Clin, 2020, 70, 7.
[2] J. M. Banales, V. Cardinale. G. Carpino, M. Marzioni, J. B. Andersen, P. Invemizzi, G. E. Lind, T. Folseraas, S. J. Forbes, L. Fouassier, A. Geier, D. F. Calvisi, J. C. Mertens, M. Trauner, A. Benedetti, L. Maroni, J. Vaquero, R. I Macias, C. Raggi. M. J. Perugorria. E. Gaudio. K. M. Boberg, J. J. Marin, D. Alvaro. Nat. Rev. Gastroenterol. Hepatol. 2016, 13, 261.
[3] S. Baroud, A. J. Sahakian, T. Sawas, A. C. Storm, J. A. Martin, B. K. Abu Dayyeh, M. Topazian, M. J. Levy, L. R. Roberts, G. J. Gores, B. T. Petersen, V. Chandrasekhara. Gastrointest. Endosc. 2021, 95, 884.
[4] J. H. Jo. C. M. Cho, J. H. Jun, M. J. Chung, T. H. Kim, D. W. Seo. J. Kim, D. H. Park, K. S. G. E. Research Group for Endoscopic Ultrasonography in. J. Gastroenterol. Hepatol. 2019, 34, 799.
[5] R. I. R. Macias, M. Komek, P. M. Rodrigues, N. A. Paiva, R. E. Castro, S. Urban. S. P. Pereira. M. Cadamuro, C. Rupp, S. H. Loosen, T. Luedde. J. M. Banales. Liver Int. 2019, 39 Suppl 1, 108.
[6] A. Tsen, M. Barbara, L. Rosenkranz. Pancreatology 2018, 18, 862.
[7] D. Marrelli, S. Caruso, C. Pedrazzani, A. Neri, E. Fernandes, M. Marini, E. Pinto, F. Roviello. Am. J. Surg. 2009, 198, 333.
[8] K. H. Son, C. B. Ahn, H. J. Kim. J. S. Kim. J. Cancer 2020, 11, 4073.
[9] J. Shen, W. Wang, J. Wu, B. Feng, W. Chen, M. Wang, J. Tang, F. Wang, F. Cheng, L. Pu, Q. Tang, X. Wang, X. Li. PLoS One 2012, 7, e47476.
[10] M. Arechederra, M. Rullan, I. Amat, D. Oyon, L. Zabalza, M. Elizalde, M. U. Latasa, M. R. Mercado, D. Ruiz-Clavijo, C. Saldaña, I. Fernández-Urién, J. Carrascosa, V. Jusué, D. Guerrero-Setas, C. Zazpe, I. González-Borja, B. Sangro, J. M. Herranz, A. Purroy, I. Gil, L. J. Nelson, J. J. Vila, M. Krawczyk, K. Zieniewicz, W. Patkowski, P. Milkiewicz, F. J. Cubero, G. Alkorta-Aranburu, M. G Fernandez-Barrena, J. M. Urman, C. Berasain, M. A. Avila. Gut 2021, 71, 1141.
[11] A. Arbelaiz, M. Azkargorta, M. Krawczvk. A. Santos-Laso, A. Lapitz. M. J. Perugorria, O. Erice, E. Gonzalez, R. Jimenez-Agiero, A. Lacasta, C. Ibarra, A. Sanchez-Campos, J. P. Jimeno, F. Lammert, P. Milkiewicz, M. Marzioni, R. I. R. Macias, J. J. G. Marin, T. Patel, G. J. Gores, I. Martinez, F. Elortza, J. M. Falcon-Perez, L. Bujanda, J. M. Banales. Hepatology 2017, 66, 1125.
[12] X. Jia, S. Lu, Z. Zeng, Q. Liu, Z. Dong, Y. Chen. Z. Zhu, Z. Hong, T. Zhang, G. Du, J. Xiang, D. Wu, W. Bai, B. Yang, Y. Li, J. Huang, H. Li, R. Safadi, Y. Lu. Hepatology 2020, 71, 893.
[13] L. Li, D. Masica, M. Ishida, C. Tomuleasa, S. Umegaki, A. N. Kalloo, C. Georgiades, V. K. Singh, M. Khashab, S. Amateau, Z. Li, P. Okolo, A. M. Lennon, P. Saxena, J. F. Geschwind, T. Schlachter, K. Hong, T. M. Pawlik. M. Canto, J. Law, R. Sharaiha, C. R. Weiss, P. Thuluvath, M. Goggins, E. J. Shin. H. Peng, V. Kumbhari. S. Hutfless, L. Zhou, E. Mezey, S. J. Meltzer, R. Karchin, F. M. Selaru. Hepatology 2014, 60, 896.
[14] H. S. Han, M. J. Kim, J. H. Han, J. Yun, H. K. Kim, Y. Yang, K. B. Kim, S. M. Park. Hepatobiliary Pancreat Dis. Int. 2020, 19, 41.
[15] V. Severino, J. M. Dumonceau, M. Delhaye, S. Moll, I. Annessi-Ramseyer, X. Robin, J. L. Frossard, A. Farina. Gastroenterology 2017, 153, 495.
[16] Q. Gou, C. Z. Zhang, Z. H. Sun, L. G. Wu, Y. Chen, Z. Q. Mo, Q. C. Mai, J. He, Z. X. Zhou, F. Shi, W Cui. W Zou, L. Lv. W H. Zhuang, R. D. Xu, W. K. Li, J. Zhang, H. W. Du, J. X. Xiang, H. Z. Wang, T Hou, S. T. Li, Y Li, X. M. Chen, Z. J. Zhou. ESMO Open 2021, 6, 100275.
[17] H. Im, H. Shao, Y. I. Park, V. M. Peterson, C. M. Castro, R. Weissleder, H. Lee. Nat. Biotechnol. 2014, 32, 490.
[18] L. Zareba, J. Szymanski, Z. Homoncik, M. Czystowska-Kuzmicz. Int. J. Mol. Sci. 2021, 22, 3651.
[19] J. McKieman, M. J. Donovan, V. O'Neill, S. Bentink, M. Noerholm, S. Belzer, J. Skog, M. W. Kattan, A. Partin. G. Andriole, G. Brown, J. T. Wei, I. M. Thompson. P. Carroll. JAMA Oncol. 2016, 2, 882.
[20] R. Xu, A. Rai, M. Chen, W. Suwakulsiri, D. W. Greening, R. J. Simpson. Nat. Rev Clin. Oncol, 2018, 15, 617.
[21] K. S. Yang, H. Im, S. Hong, I. Pergolini, A. F. Del Castillo, R. Wang, S. Clardy, C. H. Huang, C. Pille. S. Ferrone. R. Yang, C. M. Castro. H. Lee, C. F. Del Castillo, R. Weissleder. Sci. Transl. Med. 2017, 9, eaa13226.
[22] K. S. Yang, D. Ciprani, A. O'Shea, A. S. Liss, R. Yang, S. Fletcher-Mercaldo, M. Mino-Kenudson, C. Fernández-Del Castillo, R. Weissleder. Gastroenterology 2021, 160, 1345.
[23] P. Zhang, X. Zhou, M. He, Y. Shang, A. L. Tetlow, A. K. Godwin, Y. Zeng. Nat. Biomed. Eng. 2019, 3, 438.
[24] K. Liang, F. Liu, J. Fan, D. Sun, C. Liu, C. J. Lyon, D. W. Bernard, Y. Li, K. Yokoi, M. H. Katz, E. J. Koay, Z. Zhao, Y Hu. Nat. Biomed. Eng. 2017, 1, 0021.
[25] A. Hoshino, H. S. Kim. L. Bojmar. K. E. Gyan, M. Cioffi, J. Hernandez, C. P. Zambirinis, G. Rodrigues, H. Molina, S. Heissel, M. T. Mark. L. Steiner, A. Benito-Martin, S. Lucotti, A. Di Giannatale, K. Offer, M. Nakajima, C. Williams, L. Nogues, F. A. Pelissier Vatter, A. Hashimoto, A. E. Davies, D. Freitas, C. M. Kenific, Y. Ararso, W. Buehring, P. Lauritzen, Y. Ogitani, K. Sugiura, N. Takahashi, M. Aleckovic, K. A. Bailey, J. S. Jolissant. H. Wang, A. Harris, L. M. Schaeffer, G. Garcia-Santos, Z. Posner, V. P. Balachandran, Y Khakoo, G. P. Raju, A. Scherz, I. Sagi, R. Scherz-Shouval, Y. Yarden, M. Oren, M. Malladi, M. Petriccione, K. C. De Braganca, M. Donzelli, C. Fischer, S. Vitolano, G. P. Wright, L. Ganshaw, M. Marrano, A. Ahmed, J. DeStefano, E. Danzer, M. H. A. Roehrl, N. J. Lacayo, T. C. Vincent, M. R. Weiser, M. S. Brady, P. A. Meyers, L. H. Wexler, S. R. Ambati, A. J. Chou, E. K. Slotkin, S. Modak, S. S. Roberts, E. M. Basu, D. Diolaiti, B. A. Krantz, F. Cardoso, A. L. Simpson, M. Berger, C. M. Rudin, D. M. Simeone, M. Jain, C. M. Ghajar, S. K. Batra, B. Z. Stanger, J. Bui, K. A. Brown, V. K. Rajasekhar, J. H. Healey, M. de Sousa. K. Kramer, S. Sheth, J. Baisch, V. Pascual, T. E. Heaton, M. P. La Quaglia, D. J. Pisapia, R. Schwartz, H. Zhang, Y Liu, A. Shukla, L. Blavier, Y A. DeClerck, M. LaBarge, M. J. Bissell, T. C. Caffrey, P. M. Grandgenett, M. A. Hollingsworth, J. Bromberg, B. Costa-Silva, H. Peinado, Y. Kang, B. A. Garcia, E. M. O'Reilly, D. Kelsen, T. M. Trippett, D. R. Jones. I. R. Matei, W. R. Jarnagin, D. Lyden. Cell 2020, 182, 1044.
[26] S. Ferguson, K. S. Yang, P. Zelga, A. S. Liss, J. C. T. Carlson, C. F. Del Castillo, R. Weissleder. Sci. Adv. 2022, 8, eabm3453.
[27] Y. T. Kang, T. Hadlock. T. W. Lo, E. Purcell, A. Mutukuri, S. Fouladdel, M. S. Raguera. H. Fairbairn, V. Murlidhar. A. Durham, S. A. McLean. S. Nagrath. Adv. Sci. 2020, 7, 2001581.
[28] J. Wang, A. Wuethrich, A. A. Sina, R. E. Lane, L. L. Lin, Y. Wang, J. Cebon, A. Behren, M. Trau. Sci. Adv. 2020, 6, eaax3223.
[29] J. Park, J. S. Park, C. H. Huang, A. Jo, K. Cook, R. Wang, H. Y. Lin, J. Van Deun, H. Li. J. Min. L. Wang. G. Yoon, B. S. Carter, L. Balaj, G. S. Choi, C. M. Castro, R. Weissleder, H. Lee. Nat. Biomed. Eng. 2021, 5, 678.
[30] H. Shao, J. Chung, K. Lee, L. Balaj, C. Min, B. S. Carter, F. H. Hochberg, X. O. Breakefield, H. Lee, R. Weissleder. Nat. Conmun. 2015, 6, 6999.
[31] P. Zhang, X. Wu, G. Gardashova, Y. Yang, Y Zhang. L. Xu, Y Zeng. Sci. Transl. Med. 2020, 12. eaaz2878.
[32] F. Tian, S. Zhang, C. Liu, Z. Han, Y Liu, J. Deng, Y Li, X. Wu, L. Cai, L. Qin, Q. Chen, Y Yuan, Y. Liu, Y. Cong, B. Ding, Z. Jiang, J. Sun. Nat. Conmun. 2021, 12, 2536.
[33] J. Ko. N. Bhagwat, T. Black, S. S. Yee. Y J. Na, S. Fisher, J. Kim. E. L. Carpenter, B. Z. Stanger, D. Issadore. Cancer Res, 2018, 78, 3688.
[34] E. Margolis, G. Brown, A. Partin, B. Carter, J. McKieman, R. Tutrone, P. Torkler, C. Fischer, V. Tadigotla, M. Noerholm, M. J. Donovan, J. Skog. Prostate Cancer Prostatic Dis. 2022, 25, 296.
[35] D. Perocheau, L. Touramanidou, S. Gurung, P. Gissen, J. Baruteau. Br. J. Pharmacol. 2021, 178, 2375.
[36] M. N. Huda, M. Nafiujjaman, I. G. Deaguero, J. Okonkwo, M. L. Hill, T. Kim. M. Nurunnabi. ACS Biomater. Sci. Eng. 2021, 7, 2106.
[37] S. Ferguson, R. Weissleder. Adv. Biosyst. 2020, 4, e1900305.
[38] J. Zhou, Z. Wu, J. Hu, D. Yang, X. Chen, Q. Wang, J. Liu, M. Dou, W. Peng, Y Wu, W. Wang, C. Xie, M. Wang, Y Song, H. Zeng, C. Bai. Sci. Adv. 2020, 6, eabc1204.
[39] A. Morales-Kastresana. T. A. Musich, J. A. Welsh. W. Telford. T. Demberg, J. C. S. Wood, M. Bigos, C. D. Ross, A. Kachynski, A. Dean. E. J. Felton, J. Van Dyke, J. Tigges, V. Toxavidis, D. R. Parks, W. R. Overton, A. H. Kesarwala, G. J. Freeman, A. Rosner, S. P. Perfetto, L. Pasquet, M. Terabe, K. McKinnon, V. Kapoor, J. B. Trepel, A. Puri, H. Kobayashi, B. Yung, X. Chen, P. Guion, P. Choyke, S. J. Knox, I. Ghiran, M. Robert-Guroff. J. A. Berzofskv, J. C. Jones. J. Extracell. Vesicles 2019, 8, 1597603.
[40] Y Tian, M. Gong, Y. Hu, H. Liu, W. Zhang, M. Zhang, X. Hu, D. Aubert. S. Zhu, L. Wu, X. Yan. J. Extracell. Vesicles 2020, 9, 1697028.
[41] Y. Yang, G. Shen, H. Wang, H. Li, T. Zhang, N. Tao, X. Ding, H. Yu. Proc. Natl. Acad. Sci. U.S.A. 2018, 115, 10275.
[42] G. G. Daaboul, P. Gagni, L. Benussi, P. Bettotti, M. Ciani, M. Cretich, D. S. Freedman, R. Ghidoni, A. Y. Ozkumur, C. Piotto, D. Prosperi, B. Santini, M. S. Ünlü, M. Chiari. Sci. Rep. 2016, 6, 37246.
[43] M. Liebel, J. Ortega Arroyo, V. S. Beltrán, J. Osmond, A. Jo, H. Lee, R. Quidant, N. F. van Hulst. Sci. Adv. 2020, 6, eabc2508.
[44] J. Min, T. Son, J. S. Hong, P. S. Cheah, A. Wegemann, K. Murlidharan, R. Weissleder, H. Lee, H. Im. Adv. Biosyst. 2020, 4, e2000003.
[45] G. Corso, W. Heusermann, D. Trojer. A. Gorgens, E. Steib, J. Voshol, A. Graff, C. Genoud. Y. Lee. J. Hean, J. Z. Nordin, O. P. B. Wiklander, S. El Andaloussi, N. Meisner-Kober. J. Extracell. Vesicles 2019, 8, 1663043.
[46] A. M. Silva, E. Lázaro-Ibáñez, A. Gunnarsson, A. Dhande, G. Daaboul, B. Peacock, X. Osteikoetxea, N. Salmond, K. P. Friis, O. Shatnveva, N. Dekker. J. Extracell. Vesicles 2021, 10, e12130.
[47] W. Shen, K. Guo, G. B. Adkins, Q. Jiang, Y. Liu, S. Sedano, Y Duan, W. Yan, S. E. Wang, K. Bergersen, D. Worth, E. H. Wilson, W. Zhong. Angew. Chem. Int. Ed. Engl. 2018, 57, 15675.
[48] J. F. Li, C. Y. Li, R. F. Aroca. Chem. Soc. Rev. 2017, 46, 3962.
[49] J. Luan. A. Seth, R. Gupta, Z. Wang. P. Rathi, S. Cao, H. Gholami Derami, R. Tang, B. Xu, S. Achilefu, J. J. Morrissey, S. Singamaneni. Nat. Biomed. Eng. 2020, 4, 518.
[50] C. Lu, R. H. Lipson. Laser Photonics Rev. 2010, 4, 568.
[51] M. Higashi. S. Yonezawa, J. J. Ho, S. Tanaka, T. Irimura, Y. S. Kim. E. Sato. Hepatology 1999, 30, 1347.
[52] S. Y Park, S. J. Roh, Y. N. Kim, S. Z. Kim, H. S. Park, K. Y Jang, M. J. Chung, M. J. Kang, D. G. Lee, W. S. Moon. Oncol. Rep. 2009, 22, 649.
[53] A. Wang, L. Wu, J. Lin, L. Han, J. Bian, Y. Wu, S. C. Robson, L. Xue, Y Ge, X. Sang, W. Wang, H. Zhao. Nat. Commun. 2018, 9, 894.
[54] S. Rizvi, G. J. Gores. Gastroenterology 2013, 145, 1215.
[55] R. Montal, D. Sia, C. Montironi, W. Q. Leow, R. Esteban-Fabró, R. Pinyol, M. Torres-Martin, L. Bassaganyas, A. Moeini. J. Peix, L. Cabellos, M. Maeda, C. Villacorta-Martin. P. Tabrizian, L. Rodriguez-Carunchio. G. Castellano, C. Sempoux, B. Minguez. T. M. Pawlik, I. Labgaa, L. R. Roberts, M. Sole, M. I. Fiel, S. Thung, J. Fuster, S. Roavaie. A. Villanueva, M. Schwartz. J. M. Llovet. J. Hepatol. 2020, 73, 315.
[56] M. J. Perugorria, P. Olaizola, I. Labiano, A. Esparza-Baquer, M. Marzioni, J. J. G. Marin, L. Bujanda, J. M. Banales. Nat. Rev. Gastroenterol. Hepatol. 2019, 16, 121.
[57] K. Yokota, S. Serada. S. Tsujii. K. Toya, T. Takahashi. T. Matsunaga, M. Fujimoto, S. Uemura, T. Namikawa, I. Murakami, S. Kobayashi, H. Eguchi, Y Doki, K. Hanazaki, T. Naka. Mol. Cancer. Ther, 2021, 20, 1713.
[58] M. J. Gu, B. I. Jang. Pathol. Oncol. Res, 2014, 20, 655.
[59] S. K. Urban, H. Sanger, M. Krawczyk. H. Julich-Haertel, A. Willms, J. Ligocka, M. Azkargorta, T. Mocan, C. Kahlert, B. Kruk, K. Jankowski, W. Patkowski, M. Krawczyk, K. Zieniewicz, W. Holówko, L. Krupa, M. Rzucidlo, K. Gutkowski, W. Wystry chowski, R. Król, J. Raszeja-Wyszomirska, A. Slomka, R. Schwab, A. Wohler, M. A. Gonzalez-Carmona, S. Gehlert, Z. Sparchez, J. M. Banales, C. P. Strassburg. F. Lammert, P. Milkiewicz, M. Komek. Liver Int. 2020, 40, 3103.
[60] P. L. Labib, G. Goodchild, S. P. Pereira. BMC Cancer 2019, 19, 185.
[61] T. Kendall, J. Verheij, E. Gaudio, M. Evert, M. Guido, B. Goeppert, G. Carpino. Liver Int. 2019, 39 Suppl 1, 7.
[62] H. Kalra, R. J. Simpson, H. Ji, E. Aikawa, P. Altevogt. P. Askenase, V. C. Bond, F. E. Borràs, X. Breakefield, V. Budnik, E. Buzas, G. Camussi, A. Clayton, E. Cocucci, J. M. Falcon-Perez, S. Gabrielsson, Y. S. Gho, D. Gupta, H. C. Harsha, A. Hendrix, A. F. Hill, J. M. Inal, G. Jenster, E. M. Kramer-Albers. S. K. Lim, A. Llorente, J. Lötvall, A. Marcilla, L. Mincheva-Nilsson, I. Nazarenko, R. Nieuwland, E. N. Nolte-'t Hoen, A. Pandey, T. Patel, M. G. Piper, S. Pluchino, T. S. Prasad, L. Rajendran, G. Raposo, M. Record, G. E. Reid, F. Sánchez-Madrid, R. M. Schiffelers, P. Siljander, A. Stensballe, W. Stoorvogel, D. Taylor, C. Thery, H. Valadi, B. W. van Balkom, J. Vázquez, M. Vidal. M. H. Wauben, M. Yáñez-Mó, M. Zoeller, S. Mathivanan. PLoS Biol. 2012, 10, e1001450.
[63] P. J. Thul, C. Lindskog. Protein Sci. 2018, 27, 233.
[64] K. S. Yang, H. Y. Lin, C. Curley, M. W. Welch, B. M. Wolpin, H. Lee, R. Weissleder, H. Im, C. M. Castro. Adv. Biosyst. 2020, 4, e2000203.
[65] F. De Leonardis, R. A. Soref, V. M. N. Passaro. Sci. Rep. 2017, 7, 3401.
[66] J. Van Deun, A. Jo, H. Li, H. Y. Lin, R. Weissleder, H. Im, H. Lee. Adv. Biosyst, 2020, 4, e1900310.
[67] H. G. Schmidt. ACS Chem. Health Saf 2022, 29, 54.
[68] Y. Yang, H. Hwang, J. E. Im, K. Lee. S. H. Bhoo, J. S. Yoo, Y. H. Kim, J. Y. Kim. J Proteome Res. 2021, 20, 5340.
[69] T. Kwak et. al., Biosens Bioelectron. 2023 Oct. 1:237:115422.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of detecting extracellular vesicles (EVs) from cholangiocarcinoma (CCA) cells in a sample, the method comprising:
obtaining a sample, e.g., from a subject;
isolating EVs from the sample;
capturing EVs isolated from the sample on a nano-plasmonic chip bearing an array of nanostructures coated with a layer of gold; and
detecting the presence and/or levels of one or more of MUC1, EGFR, and EpCAM, wherein a higher expression level of one or more of MUC1, EGFR, and EpCAM compared to a corresponding reference level indicates that the sample includes EVs from CCA cells.

2. The method of claim 1, wherein the method further comprises immunolabeling the captured EVs using a binding ligand.

3. (canceled)

4. The method of claim 22, wherein the binding ligand is an antibody comprising a fluorophore-conjugated fluorescence primary antibody or an unconjugated primary antibody, with fluorophore-conjugated secondary antibodies labeling the primary antibody.

5. The method of claim 1, further comprising permeabilizing EV membranes for antibodies targeting proteins inside the EVs.

6. The method of claim 4, wherein the fluorophore is selected from Cy3, Cy5, AF367, AF405, AF488, AF555, AF647, DL755, and Cy7.

7. The method of claim 2, wherein the binding ligand is a nucleic acid probe comprising an aptamer, a molecular beacon, or a DNA strand.

8. The method of claim 4, wherein a presence, a level, or both a presence and a level of one or more of MUC1, EGFR, and EpCAM is determined by the fluorescence signal of the fluorophore-conjugated ligands.

9. The method of claim 1, wherein the method further comprises imaging the plasmonic chip by plasmon-enhanced multi-channel imaging.

10. The method of claim 9, wherein the plasmon-enhanced multi-channel imaging comprises amplifying fluorescent signals from the plasmonic gold nanowell arrays at multiple wavelengths to improve single EV detection sensitivity.

11. (canceled)

12. The method of claim 1, wherein the corresponding reference level is a level of expression of MUC1, EGFR, and EpCAM on EVs from a healthy subject.

13. The method of claim 1, wherein the nanostructures comprise nanoholes, nanorods, nanodisks, nanowells, nanosquares, nanopillars, or nanogrooves.

14-19. (canceled)

20. A nanowell nano-plasmonic chip for detecting target extracellular vesicles (EVs) in a sample, comprising
a substrate;
a plurality of nanowells arranged to form a periodic array of nanowells on the substrate, wherein the periodic array of nanowells is arranged and dimensioned to amplify one or more optical signals of electromagnetic radiation emitted, scattered, or reflected by EVs bound to the nanowells and/or EVs bound to the substrate near the nanowells, or to amplify one or more optical signals of electromagnetic radiation emitted, scattered, or reflected by reporter groups attached to the EVs; and
one or more affinity ligands fixed on or adjacent to the nanowells, wherein the affinity ligands selectively bind to target EVs to bind the target EVs to the nanowells or to the substrate adjacent to the nanowells.

21. The nano-plasmonic array of claim 20, wherein the one or more affinity ligands comprise polyethylene glycol (PEG).

22. The nano-plasmonic array of claim 20, wherein the optical signal comprises a fluorescent signal, a Raman signal, or dark-field scattering.

23. The nano-plasmonic array of claim 20, wherein the nanowells are coated with a layer of gold (Au).

24. A method of producing a nanowell nano-plasmonic chip, the method comprising
obtaining a semiconductor wafer with a silicon nitride (SiNx) surface layer;
applying a photoresist coating on top of the SiNx layer;
patterning a periodic nanowell array using lithography; and
depositing a layer of metal on the top of the patterned arrays,
thereby producing a nanowell nano-plasmonic chip.

25. The method of claim 24, wherein the metal layer is a layer comprising one or more of gold, silver, copper, aluminum, or platinum, and their alloys, wherein the layer is between 50 nm and 500 nm thick.

26. (canceled)

27. The method of claim 24, wherein the semiconductor wafer comprises a silicon wafer.

28. The method of claim 24, further comprising applying an anti-reflection coating (ARC) on top of the photoresist coating.

29. The method of claim 24, further comprising applying an anti-reflection coating (ARC) on top of the SiNx layer and below the photoresist coating.

30-43. (canceled)
